(19) United States
(12) Patent Application Publication
(10) Pub. No.: US 2014/0074090 A1
(43) Pub. Date: Mar. 13, 2014

(54) TISSUE WELDING USING PLASMA

(71) Applicant: IONMED LTD, Yokneam (IL)

(72) Inventors: Amnon Lam, Kibbutz Givat Oz (IL); Michael Maller, Eshchar (IL)

(21) Appl. No.: 14/116,782

(22) PCT Filed: May 9, 2012
(86) PCT No.: PCT/IL2012/050162

§ 371 Date: Nov. 11, 2013

Related U.S. Application Data
(60) Provisional application No. 61/483,971, filed on May 9, 2011.

(51) Int. Cl. A61B 18/04

(52) U.S. Cl. CPC A61B 18/042
USPC 606/49

(57) ABSTRACT

A medical device (100) for tissue welding is provided that comprises at least one processor (161) configured to regulate cold plasma production in a plasma head (102) by controlling an RF power source (141) to supply RF plasma-producing power to the plasma head.

### FIELD OF THE INVENTION

The present invention relates to an apparatus and method for tissue welding using plasma.

### BACKGROUND OF THE INVENTION

Traditional methods for closing tissue wounds or incisions include the use of glues, sutures, clips, or staples. While such techniques are generally adequate in sealing tissue wounds or incisions, they have associated problems that limit their use. For example, often lead to scar formation, infection, and a multitude of immunological responses. Tissue incompatibility with sutures, clips, or staples may cause fistulas, granulomas, and neuromas that can be painful and difficult to treat. Sutures, clips, or staples may also tend to cut through weak parenchymatous or poorly vascularized tissue. Additionally, sutures leave behind a tract that can allow for leakage of fluids and can provide a convenient entry point for a variety of organisms.

The success of traditional methods in sealing tissue wounds or incisions also is very dependent on the skill of the practitioner performing such methods, especially when microsurgery is being performed.

An alternative to traditional methods for sealing tissue wounds or incisions is the use of compositions suitable for tissue welding. By “tissue welding” it is meant that an energy source is used to excite the composition, which results in the sealing or closure of the tissue wound or incision. Typically, a tissue welding composition will be applied to the area of the tissue that requires sealing. Upon excitation by an energy source, the composition fuses to the tissue, and the bonding between the composition and the tissue allows the severed parts of the tissue to be proximal to each other, much in the same way as when sutures, staples, or clips are used. Such tissue welding compositions are absorbable within a few weeks and, therefore, do not cause tissue scar formation.

Numerous instruments are known which coagulate, seal, join, or cut tissue. Some of these devices may operate with a heating element in contact with the tissue, with an ultrasonic heater that employs frictional heating of the tissue, or with a mono- or bi-polar electrode heating system that passes current through the tissue such that the tissue is heated by virtue of its own electrical resistance.

Some devices heat the tissue to temperatures such that the tissue is either “cut” or “sealed”, as follows. When tissue is heated in excess of 100° Celsius, the tissue will be broken down and is thus, “cut”. However, when the tissue is heated to temperatures between 50° to 90° Celsius, the tissue will instead simply “seal” or “weld” to adjacent tissue. Numerous devices employing the same general principle of controlled application of a combination of heat and pressure can be used to join or “weld” adjacent tissues to produce a junction of tissues or an anastomosis of tubular tissues.

Mono-polar and bipolar probes, forceps or scissors use high frequency electrical current that passes through the tissue to be coagulated. The current passing through the tissue causes the tissue to be heated, resulting in coagulation of tissue proteins. In the mono-polar variety of these instruments, the current leaves the electrode and after passing through the tissue, returns to the generator by means of a “ground plate” which is attached or connected to a distant part of the patient's body. In a bipolar version of such an electro-surgical instrument, the electric current passes between two electrodes with the tissue being placed or held between the two electrodes.

There are many examples of such mono-polar and bipolar instruments commercially available today from companies including Valley Lab, Cabot, Meditron, Wolf, Storz and others worldwide.

In ultrasonic tissue heaters, a very high frequency (ultrasonic) vibrating element or rod is held in contact with the tissue. The rapid vibrations generate heat causing the proteins in the tissue to become coagulated.

Applying electrically generated plasma to medical application is known in the art.

For example, electrosurgery surgery is known in the art and is performed by electrical methods. Its development has been driven by the clinical need to control bleeding during surgical procedures. While heat has been used medically to control bleeding for thousands of years, the use of electricity to produce heat in tissue has only been in general use since the mid 1920's, and in flexible endoscopy since the 1970's. Electrosurgery offers at least one unique advantage over mechanical cutting and thermal application: the ability to cut and coagulate tissue at the same time. This advantage makes it the ideal surgical tool for the gastroenterologist.

Electrosurgical Generators provide the high frequency electrical energy required to perform electrosurgery and some of these are equipped with an option to use argon gas enhanced electrosurgery. Argon gas enhanced or Argon Plasma Coagulation (APC) has been in long use in the operating room setting and is used intermittently, usually for parenchymal organ surgeries.

Argon plasma equipped electrosurgery systems were adapted to be able to be used in flexible endoscopic procedures of the gut and lung.

Optical emission spectroscopy is known in the art and is commonly used to identify chemical composition and abundance of chemical species in mixtures. Plasma may excite the mixture, and the emitted fluorescence is collected and analyzed in a spectrometer.

Large amount of research was devoted to laser tissue welding. Companies such as Laser Tissue Welding Inc. (Texas, USA) have started clinical trials in 2009. This company targets for internal organs closure. Seraffix, an Israeli startup company using a robotic CO₂ laser device also started clinical trials in 2009. Laser soldering utilizes IR laser (wavelength>1 um), mostly CO₂ source, which activates thermally albumin that is applied pre activation. The laser grater advantage is its spatial accuracy which can get to micrometers resolution. However, for soldering application, the spatial accuracy is of less importance.

The main disadvantage of the laser is that its thermal activation is linearly dependent on the time it “hits” the targeted area; this means that if the laser beam stays too long on the same spot, it burns the albumin and the tissue in vicinity, performs poor adhesion and tissue necrosis.

U.S. Pat. No. 7,033,348; titled “Gelatin based on Power-gel™ as solders for Cr4+ laser tissue welding and sealing of lung air leak and fistulas in organs”; to Alfano, R. et. al; discloses a method of welding tissue, involves joining edges of tissue wound and irradiating wound with laser selected from group consisting of Cr4+ lasers, semiconductor lasers and fiber lasers where the weld strength follows the absorption spectrum of water. The use of gelatin and esterified gelatin as solders in conjunction with laser inducted tissue welding impart much stronger tensile and torque strengths than albumin solders. Selected NIR wavelength from the above lasers can improve welding and avoid thermal injury to tissue when used alone or with gelatin and esterified gelatin solders. These discoveries can be used to enhance laser tissue welding of tissues such as skin, mucous, bone, blood vessel, nerve, brain, liver, pancreas, spleen, kidney, lung, bronchus, respiratory track, urinary tract, gastrointestinal tract, or gynecologic tract and as a sealant for pulmonary air leaks and fistulas such as intestinal, rectal and urinary fistulas.

US application 20060217706; titled “Tissue welding and cutting apparatus and method”; to Lau, Liming, et. al.; discloses a surgical apparatus and methods for severing and welding tissue, in particular blood vessels. The apparatus includes an elongated shaft having a pair of relatively movable jaws at a distal end thereof. A first heating element on one of the jaws is adapted to heat up to a first temperature and form a welded region within the tissue, while a second heating element on one of the jaws is adapted to heat up to a second temperature and sever the tissue within the welded region.

U.S. Pat. No. 7,112,201; titled “Electrosurgical instrument and method of use”; to Truckai, Csaba, et. al.; discloses an electrosurgical medical device and method for creating thermal welds in engaged tissue. In one embodiment, at least one jaw of the instrument defines a tissue engagement plane carrying a conductive-resistive matrix of a conductively-doped non-conductive elastomer. The engagement surface portions thus can be described as a positive temperature coefficient material that has a unique selected decreased electrical conductance at each selected increased temperature thereof over a targeted treatment range. The conductive-resistive matrix can be engineered to bracket a targeted thermal treatment range, for example about 60° C. to 80° C., at which tissue welding can be accomplished. In one mode of operation, the engagement plane will automatically modulate and spatially localize Ohmic heating within the engaged tissue from RF energy application-across micron-scale portions of the engagement surface. In another mode of operation, a conductive-resistive matrix can induce a “wave” of RF energy density to sweep across the tissue to thereby weld tissue.

US application 20030055417; titled “Surgical system for applying ultrasonic energy to tissue”; discloses an ultrasonic surgical instrument for sealing and welding blood tissues, having wave guide moving relative to introducer and ultrasound source coupled to elongated jaws moving to selected approximate position.

U.S. Pat. No. 6,323,037; titled “Composition for tissue welding and method of use”; to Lauto, Antonio, and Poppas, Dix P.; discloses a composition for tissue welding. The composition comprises an active compound, a solvent, and an energy converter and is insoluble in physiological fluids. A method for welding a tissue is also provided. The method comprises contacting a tissue with the above composition and exciting the composition such that the tissue becomes welded.

U.S. Pat. No. 7,186,659 titled “Plasma etching method”; to Fujimoto, Kotaro and Shimada, Takeshi; discloses an etching method for etching semiconductor devices, involves introducing etching gas in etching chamber, and exciting etching gas to plasma state to etch the material.

U.S. Pat. No. 6,197,026; titled “Electrosurgical instrument”; to Farin, Gunter and Grund, Karl Ernst; discloses an electrosurgical instrument for plasma coagulation of biological tissue e.g. for treating blood clots, haemostasis, thermal devitalization or destruction of pathological tissue.

US application 20080119843; titled “Compact electrosurgery apparatuses”; to Morris, Marcia; discloses a compact electrosurgical apparatus for use in electrosurgery such as flexible endoscopy.

U.S. Pat. No. 6,890,332; titled “Electrical discharge devices and techniques for medical procedures”; to Truckai, Csaba and Shadduck; discloses a medical instrument coupled to a source for introducing a gas to controllably form and capture transient gas volumes in a microchannel structure at the working surface of the instrument that interfaces with a targeted tissue site. Each of the microchannel features of the working surface carries an electrode element coupled to the electrical source. The energy may be applied to the targeted site in either of two modes of operation, depending in part on voltage and repetition rate of energy delivery. In one mode of energy application, electrical potential is selected to cause an intense electrical arc across the transient ionized gas volumes to cause an energy-tissue interaction characterized by tissue vaporization. In another preferred mode of energy delivery, the system applies selected levels of energy to the targeted site by means of energetic plasma at the instrument working surface to cause molecular volatilization of surface macromolecules thus resulting in material removal. Both modes of operation limit collateral thermal damage to tissue volumes adjacent to the targeted site.

U.S. Pat. No. 5,083,004; titled “Spectroscopic plasma torch for microwave induced plasmas”; to Wells, Gregory and Bolton, Barbara; discloses spectroscopic plasma torch suitable for use at atmospheric pressure.

### SUMMARY OF THE INVENTION

The present invention relates to an apparatus and method for tissue welding applications using a plasma head.

In the context of the present application, the term “tissue welding” refers to procedures that cause otherwise separated tissue to be sealed, coagulated, fused, welded or otherwise joined together.

The current invention discloses a medical device for tissue welding comprising: a supply and control unit comprising: a battery providing electrical power; gas handling sub-system comprising: a gas tank storing plasma gas under high pressure; gas pressure reduction and flow control mechanism; RF circuit comprising: RF generator; RF amplifier; and RF impedance matching circuitry; a hose, transferring gas from said gas handling sub-system and RF signal from said RF circuit to a hand-held plasma head; and a plasma head capable to be held and manipulated by a single human hand, said plasma head comprising: a tip comprising: a body configured to be held by hand; a tip comprising: a plasma tube or chamber having a proximal opening and a distal opening, receiving gas through its proximal opening and providing plasma through its distal opening; and plasma exciter, exciting said gas in said plasma tube/chamber to plasma.

Plasma heads configured for deep cuts and long cuts are provided. Additionally, methods for welding deep cuts are provided.

Cuts deeper then 1 cm may be difficult to weld in one plasma application. Such cuts may be treated with repeated application of solder and plasma solidifying of the solder.

In an exemplary embodiment of the current invention, a sequence of solder deposition and plasma solidifying of the deposited solder is applied to the tissue to be welded. For example, a 0.25 to 2 seconds spray of liquid solder is directed to the tissue, followed by a 0.25 to 2 seconds application of plasma with short or no dwell duration between. The sequence then repeated. Preferably, an automatic control of the spraying and plasma generation is used to produce repeated sequence of solder spray and plasma solidification. The dual purpose solder-plasma applicator may be held stationary for welding a short deep cut, or be moved along a deep cut in one direction or in a back and forth fashion.

The current invention provides devices and methods for generating plasma using dialectic discharge using insulated electrode, and inductively excited plasma using one sided antenna coils.

The current invention provides devices and methods for efficient, time saving and convenient tissue welding using multi-head plasma applicator having a plurality of functions.

The current invention provides devices and methods for controlling the plasma and the welding process.

The current invention provides devices and methods for uniform spreading of solder to the weld area.

The current invention provides devices and methods for pre-welding disinfection of the weld area.

The current invention provides devices and methods for post-welding sealing of the weld area.

The current invention provides devices and methods for reducing bleeding of weld area.

In an exemplary embodiment, a medical device for tissue welding is provided, the device comprising: a gas source supplying gas to a plasma tip; an RF power source supplying RF power to said plasma tip; and a plasma tip comprising: a plasma tube having a proximal opening and a distal opening, receiving gas from said gas source through its proximal opening and providing plasma through its distal opening; and at least one electrode, receiving RF power from said RF source, and exciting said gas to produce said plasma, wherein said electrode is not in direct contact with said gas such that plasma is produced via dielectric breakdown and not via corona plasma production.

In some embodiments the medical device further comprising grounding electrode pad grounding said tissue.

In some embodiments the plasma tip comprises a single electrode.

In some embodiments the single electrode is in a form of a coil receiving RF power from one end only.

In some embodiments the plasma tube is electrically insulating, and said coil is located on the outer side of said plasma tube.

In an exemplary embodiment, a medical method of tissue welding is provided, the method comprises the steps of: depositing a first layer of solidified bio-compatible solder by moving over a tissue to be welded a single hand held dispenser-plasma device while: applying to the tissue to be welded, bio-compatible liquid capable of solidifying in response to application of plasma; and solidifying said bio-compatible liquid by applying plasma to said bio-compatible liquid.

In some embodiments the temperature of said plasma is less than 70 degrees Celsius.

In some embodiments the bio-compatible liquid comprises hemostatic agent.

In some embodiments the bio-compatible liquid comprises chitosan.

In some embodiments the method further comprises depositing an additional layer of solidified bio-compatible solder at a location where said first layer was deposited by repeating the step of depositing a layer of solidified bio-compatible solder.

In an exemplary embodiment, a medical method of tissue welding is provided, the method comprises the steps of: pre-welding disinfection of the tissue to be welded by applying plasma to said tissue; applying to the tissue to be welded, bio-compatible liquid capable of solidifying in response to application of plasma; and solidifying said bio-compatible liquid by applying plasma to said bio-compatible liquid.

In some embodiments the gas used for pre-welding disinfection comprises gases selected from a group consisting of N₂, O₂ and air.

In an exemplary embodiment, a medical method of tissue welding is provided, the method comprises the steps of: applying to the tissue to be welded, bio-compatible liquid capable of solidifying in response to application of plasma; and sealing said welded tissue by applying post-welding plasma to said tissue.

In some embodiments the gas used for post-welding plasma sealing comprises Argon.

In an exemplary embodiment, a medical method of tissue welding is provided, the method comprises the steps of: pre-welding disinfection of the tissue to be welded by applying plasma to said tissue; applying to the tissue to be welded, bio-compatible liquid capable of solidifying in response to application of plasma; and sealing said welded tissue by applying post-welding plasma to said tissue.

In an exemplary embodiment, a medical device for tissue welding is provided, the device comprising: a gas source; an RF power source supplying RF power; at least a first and a second plasma head; and a solder dispenser.

In some embodiments the solder dispenser is located between said first plasma head and said second plasma head.

In some embodiments the device further comprising at least a third plasma head.

In an exemplary embodiment, a medical device for tissue welding is provided, the device comprising: a gas source; a plasma head producing cold plasma; and an RF power source, supplying RF plasma producing power to said plasma head,

wherein said power producing RF power comprises a carrier frequency of 1 to 10 MHz which is modulated at frequency of 200 to 600 Hz and duty cycle of 5 to 20%.

In some embodiments the carrier is sinusoidal.

In some embodiments the modulation is an on/off modulation.

In some embodiments the plasma producing RF power further comprises a DC component.

In some embodiments the carrier of said plasma producing RF power is at the range of 100 to 500 Volts.

In some embodiments the power supply is capable of delivering to said plasma head an RF plasma ignition signal larger then 1500 Volts.

In an exemplary embodiment, a medical method of tissue welding, the method comprises the steps of: applying to the tissue to be welded, bio-compatible liquid capable of solidifying in response to application of plasma; and solidifying said bio-compatible liquid by applying plasma to said bio-compatible liquid; and controlling said plasma applied to said bio-compatible liquid by: monitoring at least one of: the electrical impedance in the plasma path, power; current or voltage; determining solidification of said bio-compatible liquid from changes in said measured impedance; and controlling RF power producing said plasma in response to said determined state of said bio-compatible liquid.

According to an aspect of the invention, the tissue to be welded does not undergo substantial changes such as denaturation, coagulation or charring. Thus, changed in impedance or other measured plasma parameters may be interpret as resulting from solidification of the solder.

Changes are limited, or at least mainly confined to the solder due to the plasma parameters used in the exemplary embodiment. Preferably the plasma used is at low temperature of less than 100° C. or less than 70° C., and applied for short time duration of several seconds for example less than 1 minute at the same location. Since the solder may be more susceptible to heat, it is affected more than the nearby tissue. Additionally and optionally, the plasma is directed at solder mainly, and thus energy is deposited mainly in it. Thus, solder temperature may be higher than the temperature of the surrounding tissue, enhancing the confinement of the changes to the solder.

In some embodiments the determining solidification of said bio-compatible liquid is from changes in said measured impedance during plasma application.

In some embodiments the controlling RF power comprises: monitoring the changes in the measured impedance; and turning off said plasma producing RF power when the change in impedance is above a preset threshold value.

In some embodiments the distance between the plasma producing head and the welded tissue is kept constant during said monitoring the changes in plasma impedance.

In an exemplary embodiment, a medical dispenser-plasma device for both applying solder solution on tissue and performing tissue welding comprising: a plasma producing head; and a dispenser for bio-compatible liquid capable of solidifying in response to application of plasma, wherein said dispenser comprises: a container holding said bio-compatible liquid; and a roller of porous material, in contact with tissue to be welded, receiving said bio-compatible liquid from said container, and capable of rotating and spreading said liquid on said tissue. In some embodiments, said dispenser-plasma device comprises a motion detector, wherein operation of at least one of said plasma producing head and said a dispenser is controlled by signals of said a motion detector

In an exemplary embodiment, a medical method for tissue welding, the method comprises: generating a first welding layer by a sequence of: spraying bio-compatible liquid onto tissue to be welded; and solidifying said bio-compatible liquid using plasma, wherein said sequence is completed within duration of less than 4 seconds; and generating a second welding layer by repeating said sequence.

In some embodiments the sequences are generated by automatic controller controlling the spraying and plasma generation.

In an exemplary embodiment, a medical method for tissue welding, the method comprises: placing a sheet of dried chitosan over a tissue to be welded; wetting said sheet of dried chitosan; and solidifying chitosan applying plasma.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

The invention is capable of other embodiments or of being practiced or carried out in various ways. Also, it is to be understood that the phraseology and terminology employed herein is for the purpose of description and should not be regarded as limiting.

In discussion of the various figures described herein below, like numbers refer to like parts. The drawings are generally not to scale. For clarity, non-essential elements were omitted from some of the drawings. Some optional parts were drawn using dashed lines.

In the drawings:

FIG. 1 schematically depicts a block diagram of plasma welding system for welding tissue according to an exemplary embodiment of the current invention.

FIG. 2A schematically depicts some details of a hand held plasma head for plasma welding according to an exemplary embodiment of the current invention.

FIG. 2B schematically depicts a disassembled plasma head comprising body and interchangeable tissue welding tip according to an exemplary embodiment of the current invention

FIG. 3 schematically depicts a cross section of a plasma welding tip according to an exemplary embodiment of the current invention.

FIG. 4A schematically depicts a cross section of a dual purpose plasma welding and ablation tip in bi-polar welding configuration, according to another exemplary embodiment of the current invention.

FIG. 4B schematically depicts a cross section of the dual purpose plasma welding and ablation tip in mono-polar ablation or coagulation configuration, according to another exemplary embodiment of the current invention.

FIG. 4C schematically depicts a cross section of a dielectric breakdown plasma tip according to another exemplary embodiment of the current invention.

FIG. 5A schematically depicts a cross section of a plasma welding tip using induction activated plasma according to yet another exemplary embodiment of the current invention.

FIG. 5B schematically depicts a cross section of a plasma welding tip using antenna coil for induction activated plasma according to yet another exemplary embodiment of the current invention.

FIG. 5C schematically depicts a large plasma head according to an exemplary embodiment of the current invention.

FIG. 5D schematically depicts a plasma head having a spiral central electrode according to an exemplary embodiment of the current invention.

FIG. 6 schematically depicts a plasma head having stand-off legs for controlling the distance of the plasma head to the treated tissue.

FIG. 7 schematically depicts a multi-head plasma applicator having a plurality of functions for increasing the efficiency of tissue welding according to an aspect of the current invention.

FIG. 8A schematically depicts block diagram of optional electrical circuited of a bi-polar plasma system according to an exemplary embodiment of the current invention.

FIG. 8B schematically depicts the electrical connections of a mono-polar plasma system according to an exemplary embodiment of the current invention.

FIG. 9A schematically depicts an electric circuit for driving a bipolar plasma head according to an exemplary embodiment of the invention.

FIG. 9B schematically depicts electronic circuit for plasma monitor.

FIG. 9C schematically depicts an RF wavefunction according to an exemplary embodiment of the current invention.

FIG. 10 schematically depicts a wiper for uniformly spreading solder solution on tissue according to another embodiment of the current invention.

FIG. 11A schematically depicts a roller device for uniformly spreading solder solution on tissue according to another embodiment of the current invention.

FIG. 11B schematically depicts a roller device for uniformly spreading solder solution on tissue according to yet another embodiment of the current invention.

FIG. 12 schematically depicts some details of a dual-function dispenser-plasma device for both applying solder solution on tissue and performing tissue welding according to yet another exemplary embodiment of the current invention.

### DESCRIPTION OF THE PREFERRED EMBODIMENTS

The present invention relates to an apparatus and method for tissue welding applications using a plasma head.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways.

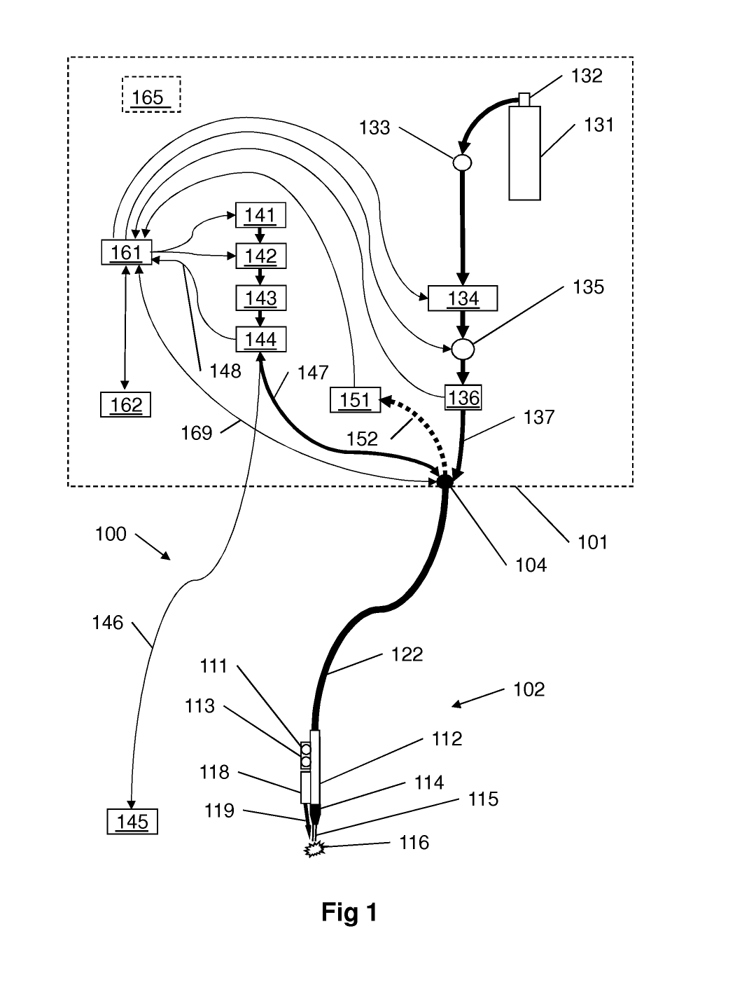

FIG. 1 schematically depicts a block diagram of plasma welding system for welding tissue according to an exemplary embodiment of the current invention.

According to an exemplary embodiment of the invention, plasma welding system 100 comprises control and supply unit 101 connected to a hand-held plasma head 102 via a flexible hose 122. Control and supply unit 101 supplies to a hand-held plasma head, 102 via a flexible hose 122 at least: gas, which is used for plasma generation, and Radio Frequency (RF) energy, for exciting the gas and creation the plasma 116.

Flexible hose 122 may optionally return to control and supply unit 101 signals indicative of welding process parameters, for example: plasma emission spectra, plasma temperature, tissue temperature, RF current, RF impedance, etc. Additionally, hose 122 may further comprise an electrical cable for transmitting commands from command switches on plasma head to control and supply unit 101.

It should be clear that Flexible hose 122 may comprise a plurality of hoses and may comprise additional tubing, electrical cables, optical fibers, etc. Similarly, it should be clear that control and supply unit 101 may be housed in one or more housing, for example, electronics and gas handling sub-units may be separately housed. Preferably, a compact and portable plasma welding system may comprise a single, compact control and supply unit

### Gas Supply Sub-System

Gas supply sub-system of plasma welding system 100 comprises at least one gas tank 131 holding pressurized gas. In the exemplary embodiment illustrated in FIG. 1, tank 131 is seen situated inside control and supply unit 101; however, tank 131 may be placed outside control and supply unit 101.

Preferably, Helium (He) gas is used due to its low breakdown voltage. Thus, low RF power is needed to produce plasma. Low RF power reduces the size and cost of the RF generator and enables operating the system using battery power, for example using the optionally rechargeable battery 165. Using gas with low breakdown voltage enables working at low plasma temperatures as needed for the welding process. However, other gases or gas mixture may be used. For example Argon (Ar) gas may be used. Specifically, other gases may be used for different applications. For example, low plasma temperature may be advantageous for plasma welding procedure, while other gases may be used for ablation of tissue, cutting tissue or coagulation. In some embodiments, a plurality of gas tanks is used holding different gases or gas mixtures.

Breakdown voltage of gases is given by Paschen's law described by the equation:

$$V = \frac{a ( pd )}{ln ( pd ) + b}$$

Where V is the breakdown voltage in Volts, p is the pressure and d is the gap distance. The constants a and b depend upon the composition of the gas. It can be seen that when working under atmospheric pressure, the breakdown voltage depends on the gas properties and the discharge gap. To reduce the breakdown voltage, the preferred gas chosen is He and the gap between the RF electrodes (or the RF electrode and the ground electrode) is minimized.

In some embodiments, a chemically active gas is used, or a chemically active component or components is added to the gas. For example, a polymerizing gas can be added to the carrying gas to enhance adhesion of the cut sidewalls. An example for such a gas is a high polymerizing gas as CHF3 or CH3F which when disassociates in the plasma enhances C and F polymer chains. Optionally, reactive gas such as O₂ is used.

Gas tank 131 may be a replaceable or disposable tank or it may be refilled on site. Preferably, gas tank 131 is equipped with a valve and connecting fitting 132 and is connected to a pressure reducing regulator 133. Regulator 133 reduces the gas high pressure in the tank to lower pressure, for example 20 to 30 psi.

Preferably, optional Mass Flow Controller (MFC) 134 is used to ensure constant and known gas flow. MFC 134 may be mechanical or electronic and is optionally controlled by controller 161 in the supply and control unit 101.

Electric solenoid valve 135 optionally controlled by controller 161 opens to allow gas flow from the gas subsystem, through gas conduit 137 to flexible hose 122.

Optionally, flexible hose is removably connected to the supply and control unit 101 by connector or plurality of connectors 104 such that several, or several types of hand held plasma heads 102 may be used with the same supply and control unit.

It should be noted that components of the gas supply sub-system may be manually controlled instead of electronically controlled by controller 161.

It was experimentally found that gas flow rate of 1 Liter per minute at 1 atm., or even substantially less, is sufficient for maintaining the plasma. Thus, a gas tank of 150 cc volume, pressurized to 200 atm. will last 30 minutes of continues operation. Such gas tank is small enough (for example a cylinder of 2 cm inner radius and 12 cm inner length) to be fitted in a compact portable unit which may be carried and used in the field. Alternatively, large gas tank may be used in stationary unit or in a unit mounted on a cart.

According to a preferred embodiment of the current invention, system 100 may be housed in a box having approximately 40×40×20 cm dimensions, wherein the plasma head is a hand held pen-like applicator connected with a hose of 1 to 2 m long.

### RF Sub-System

Supply and control unit 101 further comprises an RF sub-system for supplying Radio Frequency (RF) power for igniting and maintaining the plasma.

The RF sub-system preferably comprises an RF generator 141 followed by amplitude modulator 142. Optionally, frequency of an RF generator 141 and modulation parameters such as: modulation depth, shape and frequency of amplitude modulator 142 are controlled by controller 161. It should be noted that modern RF generators may perform both RF generation and modulation. RF signal is then amplified by RF amplifier 143 which may also be controlled by controller 161. Alternatively, pulsed DC power may be used.

Electrical power is preferably coupled to the RF input line 147 through optional impedance matching circuit 144. Preferably, RF input line 147 is a coaxial electric cable. In some embodiments, plasma is produced in “bi-polar” mode, wherein RF circuit is completed by plasma created between two closely spaced electrodes at the tip of plasma head 102.

Preferably, a grounding electrode 145, connected is attached to the patient's skin, for example to his/her hand, or attached in proximity to the plasma treated zone. Grounding electrode 145 is connected to the RF sub-system via electric cable 146. Grounding the patient is both a safety measure and it allows using the plasma head in “mono-polar” mode, wherein RF electric circuit is completed through the patient's tissue, grounding electrode 145 and electric cable 146.

According to an exemplary embodiment of the current invention RF frequency higher than 100 KHz is used, for example 1 to 20 MHz. Preferably a frequency of approximately 4 MHz is used, however lower or higher frequency may be used. According to an exemplary embodiment of the invention, RF power 0.5 to 15 Watt is used. This power level allows both tissue welding and tissue etching at a rate of 1 to 50 mm/min, however higher or lower power levels and ablation rates may be used for higher or lower rates.

Preferably, RF signal is modulated for enhancing the plasma ignition and maintenance efficiency while keeping the plasma characteristics of the carrier wave. For example plasma is generated with a carrier wave frequency of 4 MHz and 99% modulation of 1000 Hz. The plasma thus produced is “non-arcing” plasma as expected from a 4 MHz frequency but is ignited and sustained by an RF power significantly lower than needed without the modulation. However, different modulation depth or, modulation frequency and modulation envelope shape may be selected.

### Plasma Control

Impedance matching circuit 144 matches the dynamic impedance of the circuit which changes according to the plasma impedance (which varies according to the plasma conditions). Additionally, RF power level may be controlled for example by: changing the gain of amplifier 143, by using modulator 142 as an attenuator; or changing the RF power generated by generator 141. Optionally, modulation parameters and RF frequency may be changed in response to changing plasma behavior, response of the tissue or welding compound, medical procedure, etc.

Optionally, signals extracted from electric cable 169 may be used for controlling the plasma as will be explained later.

Similarly, signals extracted from impedance matching circuit 144, via electric connection 148 may also be used for controlling the plasma. Optionally, in some embodiments, processor 161 receives signal indicative of plasma process, for example by monitoring electrical plasma current or plasma impedance, for example through monitoring line 148.

In some embodiments, impedance matching circuit 144 comprises a resistor and voltage developed on said resistor is indicative of plasma current. In some embodiments, said resistor is situated within the plasma head. In some embodiments, in close proximity to the plasma electrode.

Optionally control and supply unit 101 further comprises an optical spectrometer 151. Spectrometer 151 receives light generated by plasma 116 via optical fiber 152. Optional optical fiber 152 delivers optical signals generated by the plasma 116 and indicative of the strength of the plasma and its stability, as well as ablation/welding products of said plasma to the optional optical spectrometer 151. Electrical signals from spectrometer 151 are reported to controller unit 161 and are used for analyzing the progress of the plasma welding or ablation. Optionally, spectrometer 151 comprises one or a plurality of optical filters and optical sensors.

For example, optical spectrometer 151 may detect the abundance of phosphorus (P) in the living cells which does not exist in the fat tissue, for example by monitoring one of the phosphorus wavelengths, for example at 253 nm.

Additionally or alternatively, other optical sensors (not seen in this figure) may be installed within plasma head 102 and be used for monitoring the welding or ablation progress. Said sensors receive power and report their reading to controller 161 through electric cable 169.

### Controller

Controller unit 161 may be a computer such as a PC or a laptop computer. However, controller 161 may be a DSP or other data processing device. Controller 161 receives user input and display user output through peripherals units 162 which may comprise some of: keyboard, mouse, foot pedal, and/or other input devices, a display, printer, loud speaker and/or other output devices, and optionally external storage devices and LAN or internet communication. Additionally, controller 161 may receive commands from optional user input devices 113 located on plasma head 102 via electric cable 169.

It should be noted however, that components of the RF sub-system may be manually controlled instead of electronically controlled by controller 161. In such embodiments, controller 161 may have limited function or missing.

In the case of a Portable device, the RF system is miniaturized using solid state devices to generate the RF and to control the process. With average RF power of 5 W, Energy conversion efficiency of 33.3% of the amplifier, and low energy consumption of the controller, generator and sensor, for example a standard Lithium 9V battery 165, having capacity average of 1200 mAh should last for 30 min. Thus, battery size is compatible with compact portable unit. In some embodiments, battery 165 is a rechargeable battery while in other embodiments, battery 165 is replaceable, and in yet other embodiments, power is supplied by plugging the power outlet.

### Plasma Head

According to an exemplary embodiment of the current invention, the plasma welding unit comprises a plasma head 102 connected to control and supply unit 101 connected to a hand-held plasma head 102 via a flexible hose 122. Typical dimensions for the pen-like plasma head 102 may be a length of approximately 15 cm and diameter of 1 to 2 cm.

In some embodiments, hose 122 is permanently connected to the control and supply unit 101, however, in other embodiments, hose 122 may be detached from control and supply unit 101 at hose connector 104. It should be noted that connector 104 may comprise a plurality of connectors for: gas supply tubing, RF line, electronic cable, and the optical fibers. Preferably, connector 104 is a quick release connector enabling to quickly replace the hose and the plasma head. Replacing plasma head may be useful for changing type of head, and for replacing the head with a new sterile head before each procedure. Optionally the hose and head are disposable. Alternatively, hose and head are sterilizable. In some embodiments the hose is connected to the head using a connector so that only the head is replaceable. In yet other embodiments, only the tip assembly 114 of the plasma head is replaceable.

Plasma head 102 comprises a body 112, adapted to be hand held. Optionally head 102 comprises control switch or switches 111 which are used by the operator for controlling the operation of system 100, for example by turning on or off or adjusting the gas flow, turning on or off or adjusting the RF power, providing composition for tissue welding, etc. Additionally, head 102 optionally comprises indicator or indicators 113, such as LEDs indicating status of system 100, for example gas flow, RF power, etc.

Additionally, plasma head 102 may comprise an injector 118 for injecting composition 250 for tissue welding, for example albumin solution which may be injected into a gap, cut or a discontinuation 260 in the tissue 270 and used as solder when activated and solidified by the plasma. Injector 118 preferably injects the tissue welding composition through a nozzle 119 which preferably terminates near the distal end of plasma tube 115. Optionally, the injector is located outside the body 112 of plasma head 102, and nozzle 119 is connected to a tube leading to the injector. In some embodiments, the injector is located within the supply and control unit 101, and is optionally activated using one of the switches 111.

It should be noted that other optional features or elements may be added to system 100, or used in combination with the system within the scope of the current invention. Similarly, some features or elements may be absent. Specifically, some of the elements are depicted below.

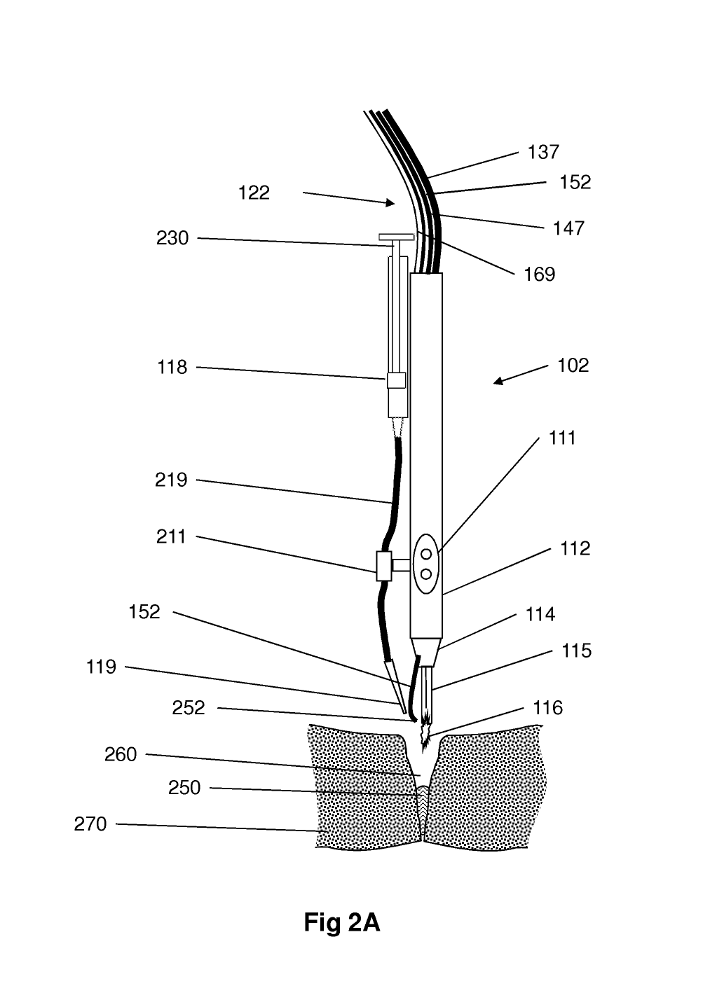

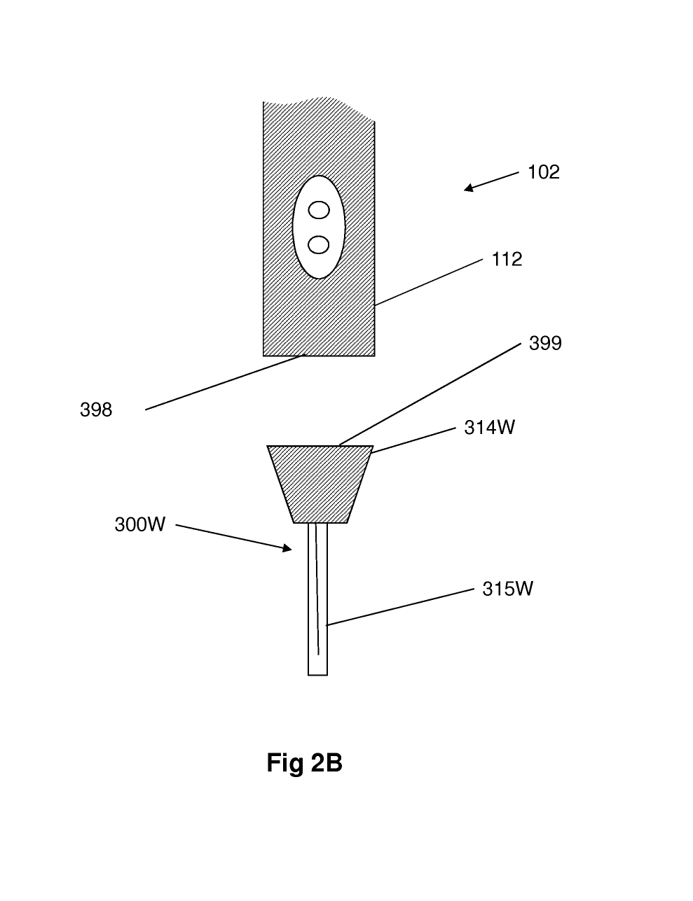

FIG. 2A schematically depicts some details of a hand held plasma head 102 for plasma welding according to an exemplary embodiment of the current invention.

In this figure, the components of hose 122, namely gas line 137, optical fiber 152, RF cable 147 and electric cable 169 are seen separately, however it should be noted that preferably all these components are housed within a common flexible shroud.

In the depicted embodiment, injector 118 is attached to, or housed inside body 112 of head 102. For example, injector 118 may be a syringe with solder solution having a spring loaded piston 230. Injector 118 is connected to nozzle 119 via solder tube 219 interrupted by mechanical or electrical valve 211 such that opening valve 211 enables application of tissue welding compound through nozzle 19 to the tissue to be welded.

Alternatively, the injector 118 may be mechanically or electrically activated to supply a predetermined amount of welding compound when it is activated. Optionally, injector 118 may comprise an electrically activated pump configured to supply welding compound at predetermined rate when it is activated.

In a proffered embodiment of the current invention, the composition 250 for tissue welding is albumin solution. Preferably, high concentration Albumin is required. Albumin may be purchased from an albumin supplier, for example from Sigma-Aldrich or Equitech-Bio, in a powder state. The albumin is mixed with sterile water to the concentration needed, for example 50% w/v.

Only small amount of albumin is needed, for example a 5 cm cut may require 5 grams of albumin at cost of $0.5 to 2.5 per gram, depending on the amount purchased.

The use of albumin as a “biological glue” is based on an albumin which when is being activated, gets denaturized and “sticks” to the surfaces in vicinity. Most of the data about using albumin as “glue” was gathered during 15 years of research done on tissue soldering using laser.

Albumin refers generally to any protein with water solubility, which is moderately soluble in concentrated salt solutions, and experiences heat coagulation (protein denaturation). The most well-known type of albumin is the serum albumin in the blood. Serum albumin is the most abundant blood plasma protein and is produced in the liver and forms a large proportion of all plasma protein. The human version is human serum albumin, and it normally constitutes about 60% of human plasma protein.

Most used albumins for soldering applications (laser) are bovine serum albumin—BSA (cattle) and human albumin. The albumin before denaturation is formed mainly in α-helix structure. It is assumed that the chemical arrangement is based mainly on electrical bond (hydrogen bonds) which gives the electric potential used by the plasma an important role.

Optionally, “custom made” solder may be developed and fitted to the plasma process characteristics.

Optical fiber 152 preferably terminates at distal end 252 located near the distal end of plasma tube 115 so that light generated by plasma 116 enters the distal end 252 of the optical fiber 152. Optionally, distal end 252 of the optical fiber 152 comprises light collection optics (not seen in this figure for clarity) for enhancing light collection efficiency and increasing signal of spectrometer 151. One problem encountered during tissue welding is overheating and even charring of the welding area. Using spectrometer 151 for monitoring the welding process may insure that the temperature stays within the safe limits.

FIG. 2B schematically depicts a disassembled plasma head 102 comprising body 112 and interchangeable tissue welding tip 300W according to an exemplary embodiment of the current invention

In this exemplary embodiment, interchangeable tip 300W is comprises connector 314W and plasma welding tube 315W. Connector 314W connects gas conduit and RF cabling in body 112 to gas channel and RF electrodes in plasma welding tube 315W. Preferably, the connection is a quick release type. For simplicity, fiber optic connection is not seen in this figure. However, optional optical fiber 152 may simply extend from body 112 for example trough a slit in connector 314W. Alternatively, an optical connector may be used with a short section of fiber. Alternatively, plasma welding tube 315W is made of transparent material such as glass, quartz, sapphire etc, and used for light collection instead of the last section of fiber 152. In this case, collected light may be confined in the transparent tube by total internal reflection, as in clad-less fiber, or a light reflecting layer may be added to the side of the tube, for example metallic or dielectric reflective coating. Light thus collected is transferred to the optical fiber in body 112. For simplicity, nozzle 119 is not seen in this figure.

It should be noted, that mating interfaces 398 and 399 on body and tip respectively may comprises of electrical connection such as contacts or plugs for transmitting electrical signals between the body and tip, gas connection that may comprise “O” ring or other gas seal, and fasteners to join the two parts.

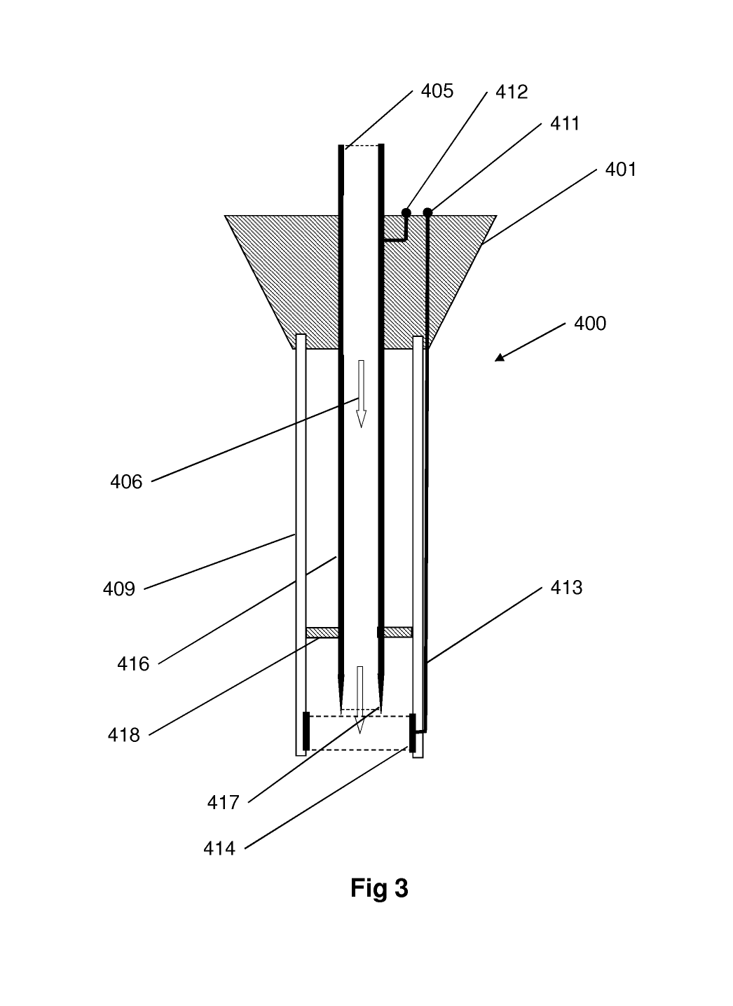

FIG. 3 schematically depicts a cross section of a plasma welding tip 400 according to an exemplary embodiment of the current invention.

For simplicity, non essential details (some already depicted in other drawings) are not depicted in this figure.

Tip 400 comprises a base 401, capable of connecting to body 112 of a plasma head. Preferably, using a quick release connector preferably having a fastener (not seen in this figure) to hold the tip in place. Tip 400 receives RF power from RF (optionally a coaxial) cable in body 112 via contacts 412 and 411. Preferably, contact 412 is connected to the central conductor of the RF cable, while contact 411 is connected to the outer conductor of said coaxial cable. Additionally, tip 400 receives gas flow 406 from gas tube in body 112 of plasma head via gas input opening 405 of central gas tube 416.

Central gas tube 416 is preferably thin metallic tube that acts also as central electrode for bi-polar plasma production. Preferably, central tube is sharpened and optionally serrated at its distal end 417 to enhance plasma production and reduce the voltage needed for ionization. Central tube 416 is held centrally to outer tube 409 using spacer 418. Outer tube 407 is preferably a thin wall tube made of non-conducting material such as glass, ceramics, plastic or quartz. A transparent outer tube enables easy visual confirmation of the plasma ignition. An annular RF grounding electrode 414 is connected to the RF cable in body 112 via return line 413 and contact 411. It should be noted that while return line 413 is seen in this figure on the outside of outer tube 404, it may be positioned inside said outer tube as long as it is properly insulated from inner tube 416.

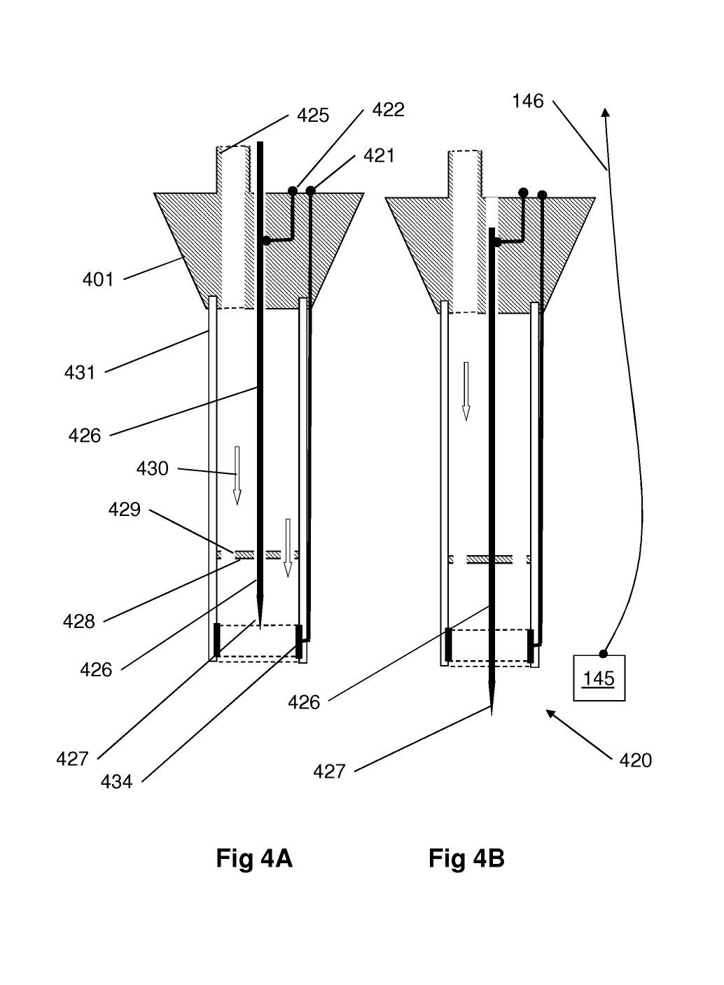

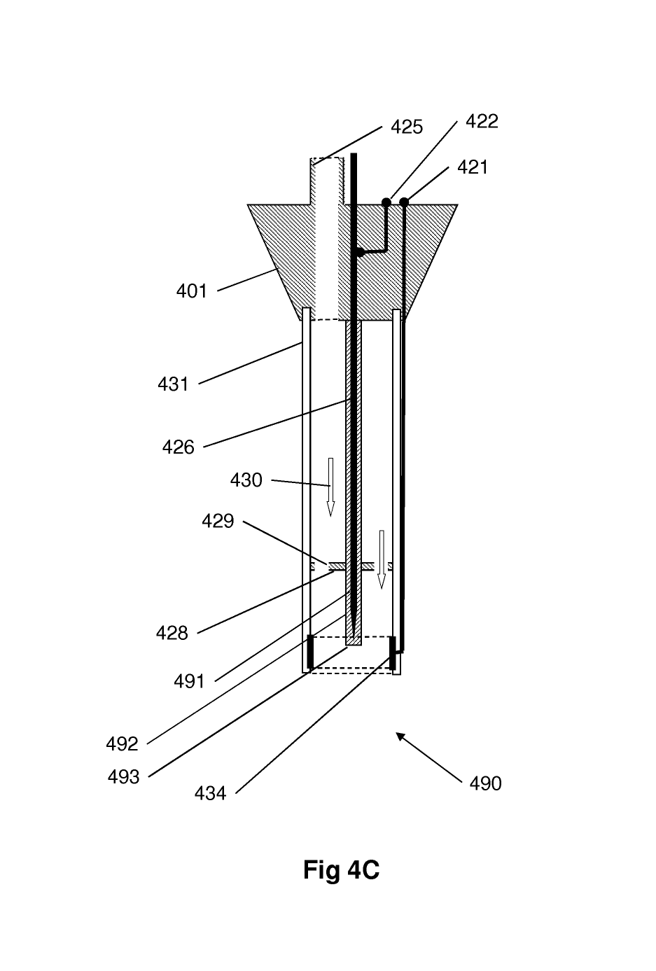

FIG. 4A schematically depicts a cross section of a dual purpose plasma welding and ablation tip 420 in bi-polar welding configuration, according to another exemplary embodiment of the current invention.

For simplicity, non essential details (some already depicted in other drawings) are not depicted in this figure. For simplicity, some parts that were already explained may not be marked in this figure.

Tip 420 comprises a base 401 (not marked in the figure), capable of connecting to body 112 of a plasma head. Preferably, using a quick release connector preferably having a fastener (not seen in this figure) to hold the tip in place. Tip 420 receives RF power from RF (optionally a coaxial) cable in body 112 via contacts 422 and 421. Preferably, contact 422 is connected to the central conductor of the RF cable, while contact 411 is connected to the outer conductor of said coaxial cable. Additionally, tip 420 receives gas flow 406 from gas tube in body 112 of plasma head via gas input opening 425 which is opened to lumen of outer tube 431.

In contrast to tip 400, tip 420 comprises a central electrode 426 instead of central gas tube 416. Central electrode 426 is preferably thin metallic rode acting as the central electrode for bi-polar plasma production. Preferably, central electrode is sharpened at its distal end 427 to enhance plasma production and reduce the voltage needed for ionization. Central electrode 426 is held centrally to outer tube 431 using spacer 428 having openings 429 to allow gas flow 430. Outer tube 431 is preferably a thin wall tube made of non-conducting material such as glass, ceramics, plastic or quartz. A transparent outer tube enables easy visual confirmation of the plasma ignition. Similarly to tip 400, an annular RF grounding electrode is connected to the RF cable in body via return line and RF connector contact 421. It should be noted that while the return line is seen in this figure on the outside of outer tube 404, it may be positioned inside said outer tube as long as it is properly insulated from inner tube 416.

FIG. 4B schematically depicts a cross section of the dual purpose plasma welding and ablation tip 420 in mono-polar ablation or coagulation configuration, according to another exemplary embodiment of the current invention.

For simplicity, non essential details (some already depicted in other drawings) are not depicted in this figure. For simplicity, some parts that were already explained may not be marked in this figure.

As depicted in this figure, central electrode 426 is pushed forward, using a mechanical lever or an electrical solenoid optionally located within body 112 of plasma head (not seen in this figure), until its distal end 427 is outside outer tube 431. In this configuration, RF circuit is completed via grounding pad 145. Preferably, RF power to annular grounding electrode 434 is turned off. However, central electrode 426 may be insulated along it length and exposed only at its tip 427. In this case, most of the current will flow through pad 145 even if annular electrode 434 is connected to the RF circuit.

FIG. 4C schematically depicts a cross section of a dielectric breakdown plasma tip 490, according to another exemplary embodiment of the current invention.

For simplicity, non essential details (some already depicted in other drawings) are not depicted in this figure. For simplicity, some parts that were already explained may not be marked in this figure.

In contrast to previously depicted tips, tip 490, comprises a central electrode 491 which is covered with electric insulator 492 such that plasma created near the tip 993 of the insulated electrode is due to dielectric breakdown of the gas. For example, a multi-layer insulation may be used, where inner layer provides most of the dielectric strength, and outer layer the environmental stability.

Electric insulator 492 may be a dielectric coating on electrode 491, or separate insulator shape as a closed end tube. Glass, quarts, ceramic, polymeric material (such as Polyimide or Mylar), or other materials may be used. Preferably, the selected material or materials are capable of withstanding the high electric fields near the tip 493, and the corrosive environment of the created plasma.

End of electrode 491 may be sharpened to increase the electric field near its point or blunt. Optionally, insulator 293 is limited to the distal part of electrode 492 and extends long enough to ensure that the plasma is created by dielectric breakdown.

It should be noted that although dielectric breakdown plasma tip 490 is depicted herein with an annular grounding electrode 434, annular grounding electrode 434 may be missing for mono-polar operation. Additionally, features depicted in FIGS. 4A and 4B may optionally be implemented, for example translation of the insulated electrode and grounding pad 145. It also should be noted that insulation may be used with other electrodes such as the ring 434 or coils 467, 487 and 856 of FIGS. (5A, 4B, and 5D respectively) or other electrodes and in other embodiments. Glass, quarts, ceramic, polymeric material (such as Polyimide or Mylar), or other materials may be used for insulation.

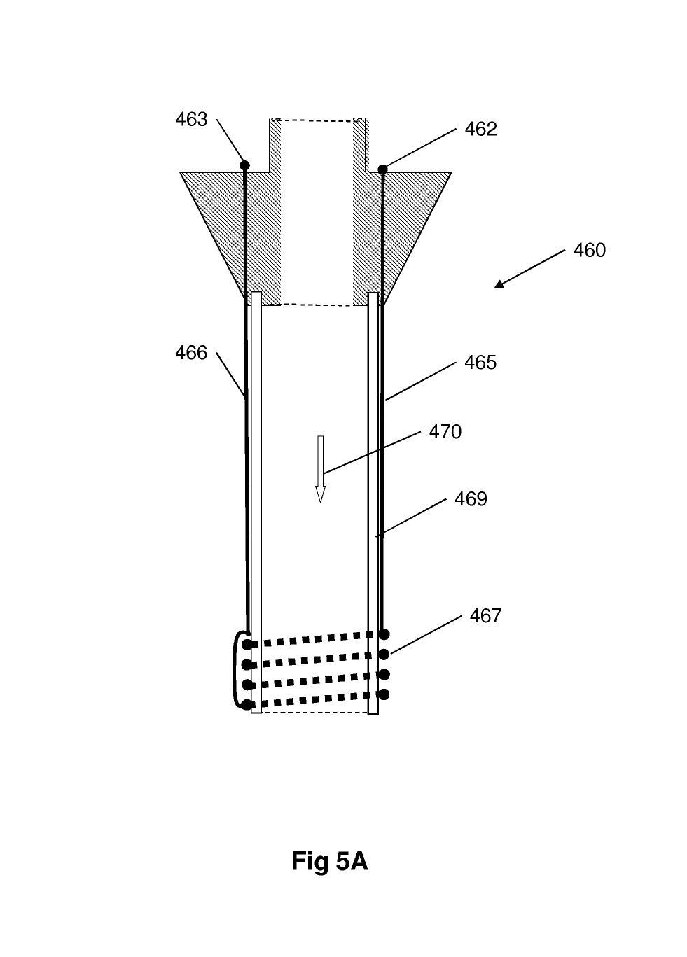

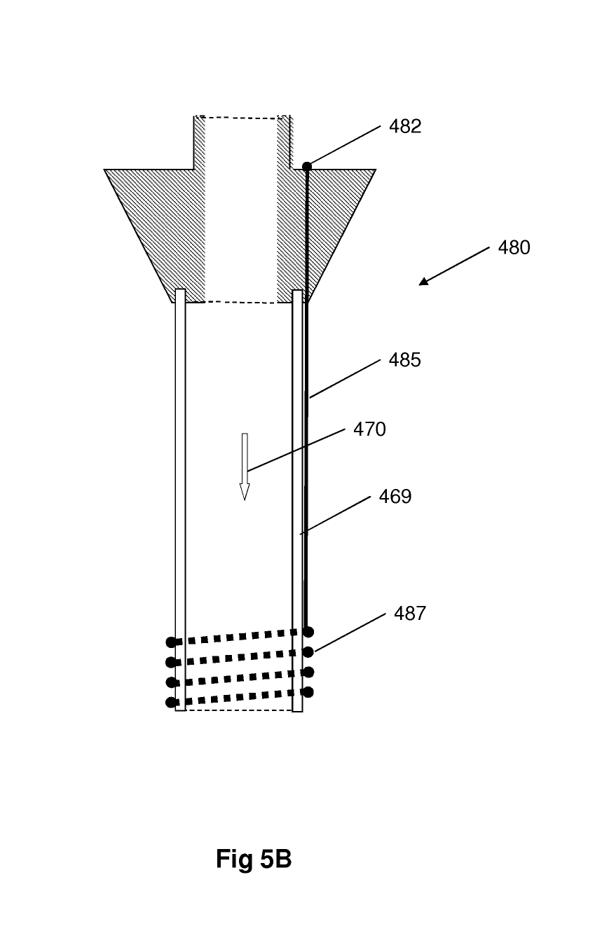

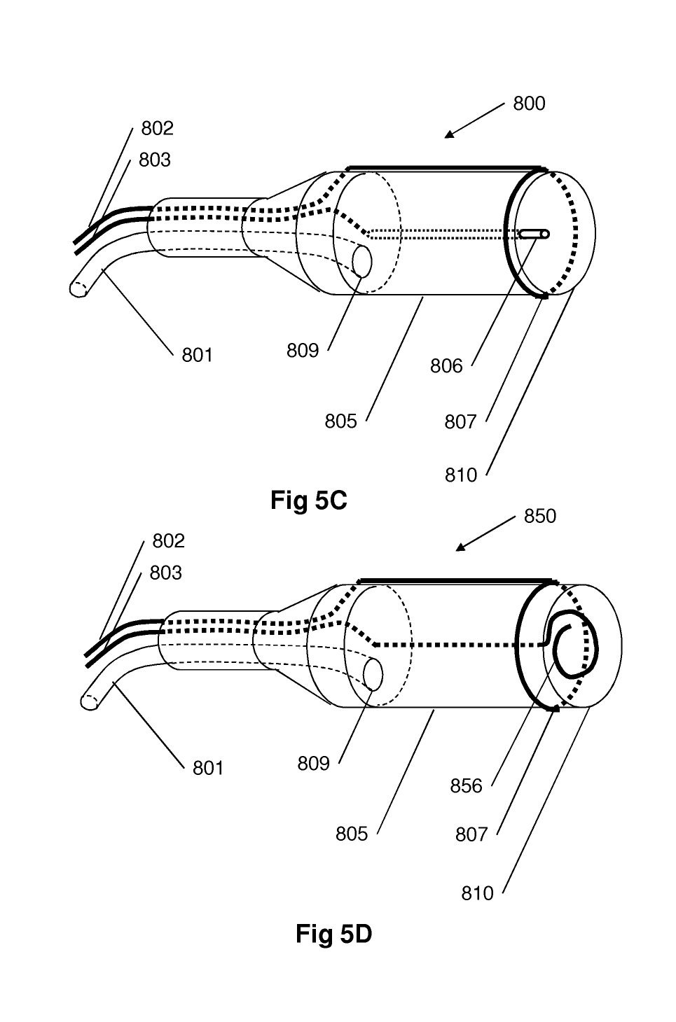

FIG. 5A schematically depicts a cross section of a plasma welding tip 460 using induction activated plasma according to yet another exemplary embodiment of the current invention.

For simplicity, non essential details (some already depicted in other drawings) are not depicted in this figure. For simplicity, some parts that were already explained may not be marked in this figure.

In contrast to tips 400, 420 and 440, RF power supplies to tip 460 via contacts 462 and 463 is connected via lines 465 and 466 to a coil 467 wound around outer tube 469. Coil 469 is preferably part of a tuned resonance circuit which may be a part of the impedance matching circuit. Alternatively, coil 469 acts as an RF antenna, not connected at its distal end) RF current in coil 467 excites the gas flow 470 in outer tube 469 and thus creates plasma. In some embodiments, number or turns in coil 469 is limited, for example only few turns, and optionally as few as 1, 1.5 or 2 turns.

In this configuration, gas flow 470 in outer tube 469 is uninterrupted, thus larger flow may be achieved, or thinner tube may be used. Although lines 465 and 466 and coil 467 are seen on the outer side of outer tube 469, it should be noted the any of them can be placed on the anterior of said tube.

FIG. 5B schematically depicts a cross section of a plasma welding tip using antenna coil 480 for induction activated plasma according to yet another exemplary embodiment of the current invention.

In contrast to coil 467 of tip 460, antenna coil 487 is connected to RF power via a single contact 482 and line 485, thus creating Inductive Coupled Plasma (ICP).

Optionally, antenna coil 487 is frequency tuned to the RF operation frequency.

FIG. 5C schematically depicts a large plasma head 800 according to an exemplary embodiment of the current invention.

Plasma head 800 receives gas through input gas pipe 801 and RF power through wires 802 and 803. Optionally, wires 802 and 803 terminates in a connector, and optionally gas pipe 801 is also detachable such that large plasma head 800 can be disconnected and replaced.

Plasma head 800 comprises a tube 805, preferably made of thin glass and having a diameter of 6 to 10 mm. Gas from pipe 801 enters tube 805 through opening 809 and exit through distal opening 810 as plasma directed towards the treated tissue. A central electrode 806 acts as a first plasma electrode, while a ring shaped electrode 807, acts as a second plasma electrode.

Preferably, ring shaped electrode 807 is placed on outside surface of tube 805. Preferably, central electrode 806 is covered with thin electrically insulating material. It was found that insulating the central electrode 806 improves plasma welding process and uniformity.

In a similar embodiment, central electrode 806 is missing. Optionally, outer electrode 807 is replaced with a coil for inductively exciting the plasma.

FIG. 5D schematically depicts a plasma head having a spiral central electrode 850 according to an exemplary embodiment of the current invention.

In contrast to previous embodiments, central electrode 806 of head 850 has a spiral shape. The spiral shape electrode creates magnetic field at center of the spiral, thus creating Inductive Coupled Plasma (ICP).

Electrode 856 may act as a coil antenna similar to antenna coil 487 of FIG. 5B.

Optionally, electrode 856 is frequency tuned to the RF operation frequency. Optionally shaped electrode 807 is missing and plasma is created by radiation of RF power from the electrode 856.

It should be noted that a large plasma heads 800 and 850 may use other plasma excitation methods depicted herein or known in the art.

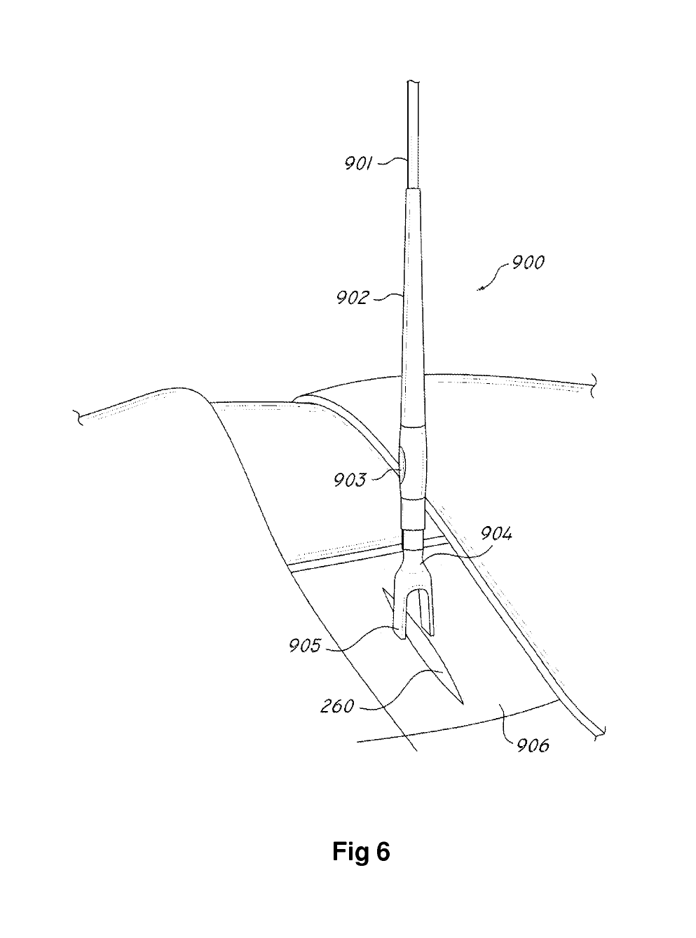

FIG. 6 schematically depicts a plasma head 900 having stand-off legs 905 for controlling the distance of the plasma head to the treated tissue.

Plasma head 900 receives RF power and gas supply via hose 901 connected to the body 902 at the proximal end of the body 902. The body 902 shaped to be hand held and to be manipulated by the user. The plasma head 900 further comprises a plasma tube 904 near the distal end of the body 902. Plasma is generated at the plasma tube 904 and exits toward the cut 260 in the patient's skin 906. To assist the user in keeping the opening of plasma tube 804 at the correct distance from the skin 906, at least one, and preferably two stand-off legs 905 protrude from the body 902 of head 900. By resting the legs 905 against the skin 906, proper distance is easily maintained.

For welding deep cuts using plasma head 900 or other heads depicted herein or known in the art, Albumin or other welding material may be applied first to the depth of the cut and cured creating a partial weld of the lower part of the cut. A second layer is then applied and the welding operation is repeated. As many applications as needed may be used to fully close the deep cut.

Optionally, a commercially available Albumin solution may be used. Commercially available solutions are of lower concentration than the ˜50% the mentioned above. However, as plasma is applied, the solution dry out and may become more concentrated. Optionally, deeper parts of the cut, which are subjected to lesser stresses may not need the full strength of the weld and may be welded with lower concentration solution.

In some embodiments of the invention, hemostasis material may be added to the solder to suppress bleeding. Material such as chitosan may be added to the solder for achieving blood coagulation when the solder is applied to the cut. This method increases the solder resistivity to bleeding or fluids secretion from the wound may melt the solder. By adding chitosan or other hemostatic agent the solder may becomes more stable.

In some embodiments, the solder can be in a form of a sheet of dissolved chitosan that was dried in a form of sheet. Preferably, these sheets may have the thickness of 20 to 300 micro meters.

Chitosan is a material extracted from shrimp's shells. It is used for hemostasis in trauma and other medical fields. Commercially, Chitosan comes in powder form but can be dissolved into a solution for example in acidic environment and added to the solder solution.

In some embodiments, acid with low Ph value such as acetic acid, or base fluid with high Ph value such as sodium hydroxide may be added to the albumin achieving stronger welding and higher stability.

In some applications, a thin sheet of 20 to 300 micro meters of dissolved and dried chitosan is placed directly over a cut to be welded. The sheet is moisten, for example by spraying water or water solution. The wet chitosan adheres to the cut and is used as solder by solidifying it using plasma application.

In some embodiments, tissue welding further comprises at least one additional step: Pre-welding plasma disinfection, and Post-welding wound sealing.

In the optional pre-welding plasma disinfection stage, plasma is applied to the tissue, in the cut and optionally to the surrounding tissue. No solder is applied at this stage.

Plasma disinfection activity may be enhanced by increasing the production of free radicals, for example by replacing the tissue welding gas. For example, gas mixture may be replaced by air or other gas or gas mixture such as Nitrogen (N₂) or Oxygen (O₂). Alternatively, gases such as N₂, O₂ or air or may be added to the tissue welding gas.

Alternatively, additionally or optionally, plasma conditions may be different during the pre-welding disinfection stage. For example, pre-welding disinfection stage may use ion bombardment and high voltage by using low gas flow and non insulated electrode (CCP—capacitive coupled plasma configuration).

Pre-welding plasma application may optionally be used for coagulation of the tissue surface, for stopping bleeding, for ablation of dead or scar tissue which may be present and interfere with the welding process, for re-shaping the tissue by ablation, or a combination thereof.

In the optional post-welding plasma sealing stage, plasma is applied to the already welded cut, over the cut and optionally to the surrounding tissue. No solder is applied at this stage. Sealing the wound using plasma application may enhance the integrity of the weld and may prevent infection.

Plasma sealing activity may be enhanced by increasing the ion bombardment, for example by replacing the tissue welding gas. For example, higher atomic mass gas as Argon may be used.

Alternatively, additionally or optionally, plasma conditions may be different during the post-welding sealing stage. For example, post-welding sealing stage may use ion bombardment and high voltage by using low gas flow and non insulated electrode (CCP—capacitive coupled plasma configuration).

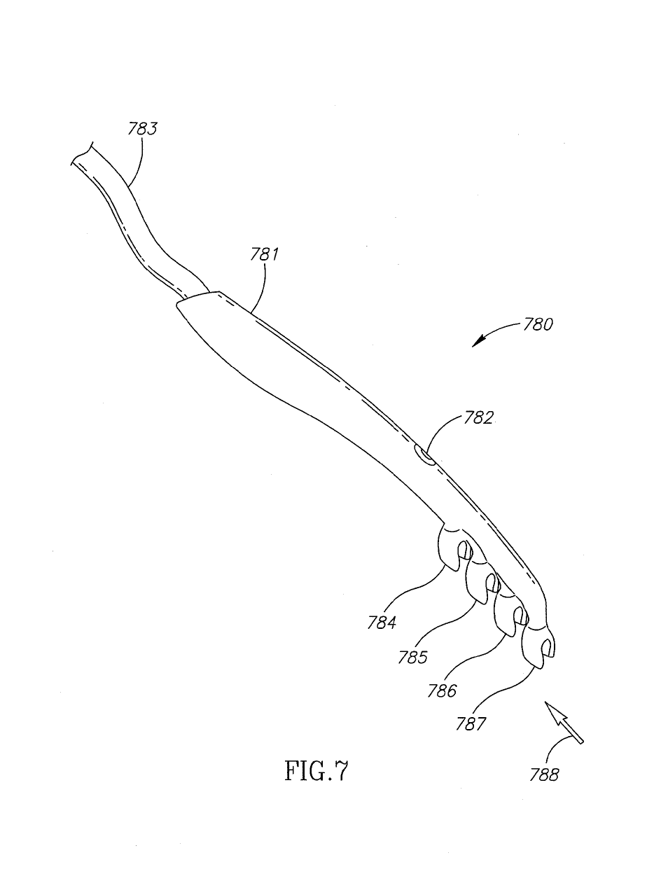

FIG. 7 schematically depicts a multi-head plasma applicator 780 having a plurality of functions for increasing the efficiency of tissue welding according to an aspect of the current invention.

Multi-head plasma applicator 780 comprises a handle 781 for manual manipulation by the user. Handle 781 may comprise indicators for indicating to the user the status and the mode of operation of the Multi-head plasma applicator. Handle 781 may comprise control switch 782 (or a plurality of switches) used by the to control the mode of operation of the Multi-head plasma applicator.

Multi-head plasma applicator 780 receives gas and RF power via cable 783 connected to a controller box such as control and supply unit 101.

Multi-head plasma applicator 780 further comprises at least two of heads 784, 785, 787, and 787.

In an exemplary embodiment of the invention, all heads 784, 785, 787, and 787 are connected to handle 782 in line such that when the applicator 780 is moved over the tissue in the direction denote by arrow 788, heads 784, 785, 787, and 787 traverses a point on the tissue in that order.

In the depicted exemplary embodiment, head 784 is a plasma disinfection head used for pre-welding disinfection for example as disclosed herein.

In the depicted exemplary embodiment, head 785 is an albumin applicator used for applying albumin or other tissue welding solution for example as disclosed herein.

In the depicted exemplary embodiment, head 786 is a plasma head used for plasma tissue welding for example as disclosed herein.

In the depicted exemplary embodiment, head 787 is a plasma head used for plasma tissue sealing for example as disclosed herein.

It should be noted that the order of the heads may be reversed and the direction of applicator 780 reversed as well.

It also should be noted that identical plasma heads may perform different functions such as disinfection, welding and sealing by optionally changing the operation parameters such as one or few of: RF power, gas flow, gas mixture and distance to the tissue. Optionally, the function of the different heads is user selectable, for example depending on the direction of motion.

For example, a symmetric multi-head plasma applicator with two, optionally identical plasma heads with an solder applicator between them may be used for disinfecting, applying solder, and welding at the same motion of the multi-head plasma applicator. The same multi-head plasma applicator may be used in a reverse motion. The optional step of sealing may be done with a second motion of the same multi-head plasma applicator with one of the plasma heads and the solder applicator head disabled. Alternatively, a single head plasma sealing device may be used.

A man skilled in the art of medical devices manufacturing may construct other combinations of heads in a multi-head plasma applicator. For example, a first multi-head plasma applicator with a plasma head and solder application head for disinfection and applying solder the tissue may be followed by a multi-head plasma applicator with a two plasma heads for welding and sealing.

It should be made clear that FIG. 7 is to be viewed only as a non-limiting illustration of a multi-function plasma treatment apparatus.

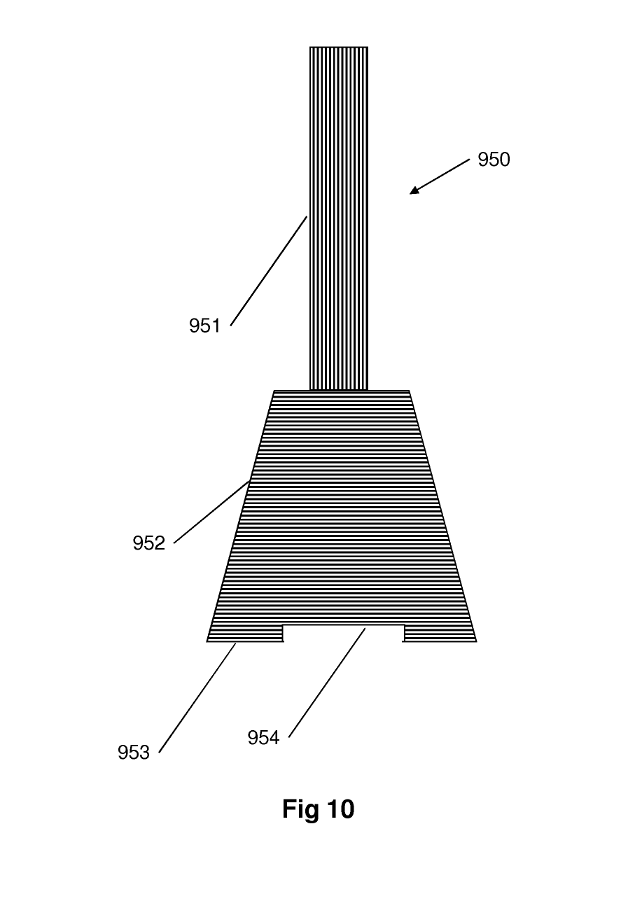

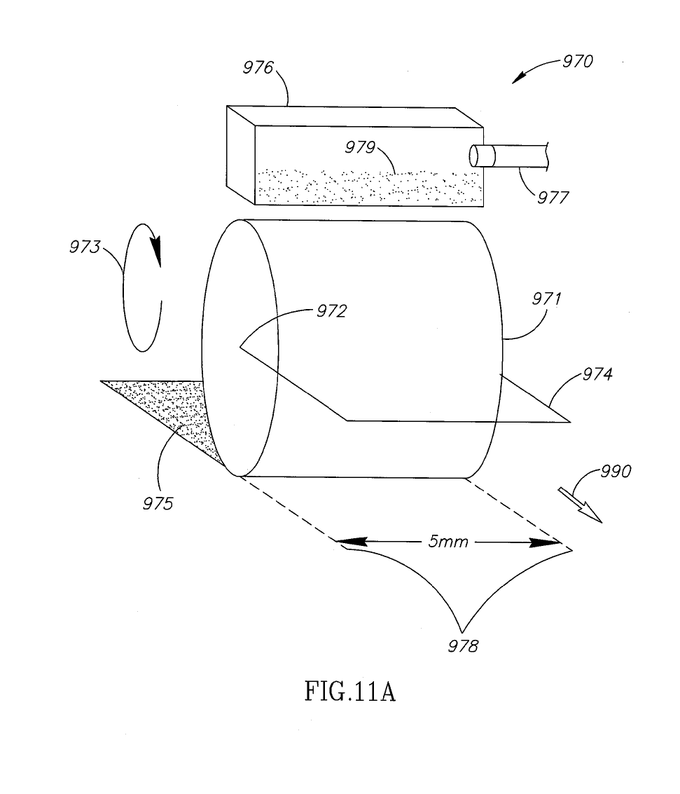

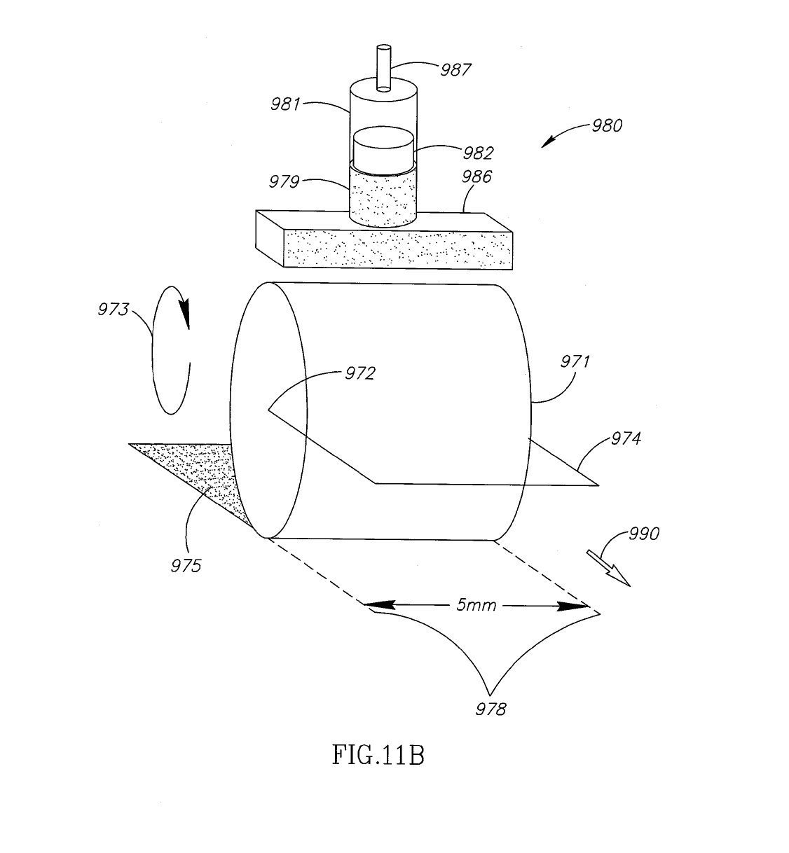

For example, the hand-held plasma head 102 of FIG. 2A may be viewed as a multi-head tool 780, for example a dual-heads tool, capable of applying solder solution and plasma to the tissue in a synchronized manner. Such a dual-head tool may comprise any solder solution dispenser such as: a solution spray dispenser, one of the more rollers, for example as depicted by numerals 770 and/or 780 depicted in FIG. 11A or 11B, a dispenser combined with wiper, for example as depicted by numeral 950 of FIG. 10, or another solution dispenser known in the art and synchronized with any of the plasma heads which are depicted herein, known in the art. This dual head tool may optionally be combined with stand-off legs 905 seen in FIG. 6. Using such dual-function tool, the user may apply solder solution and solidify the applied solution in one motion over the cut. For deep cut, several passages over the cut may be needed.

Other combinations of head numbers and types may be constructed and are within the general scope of the current invention.

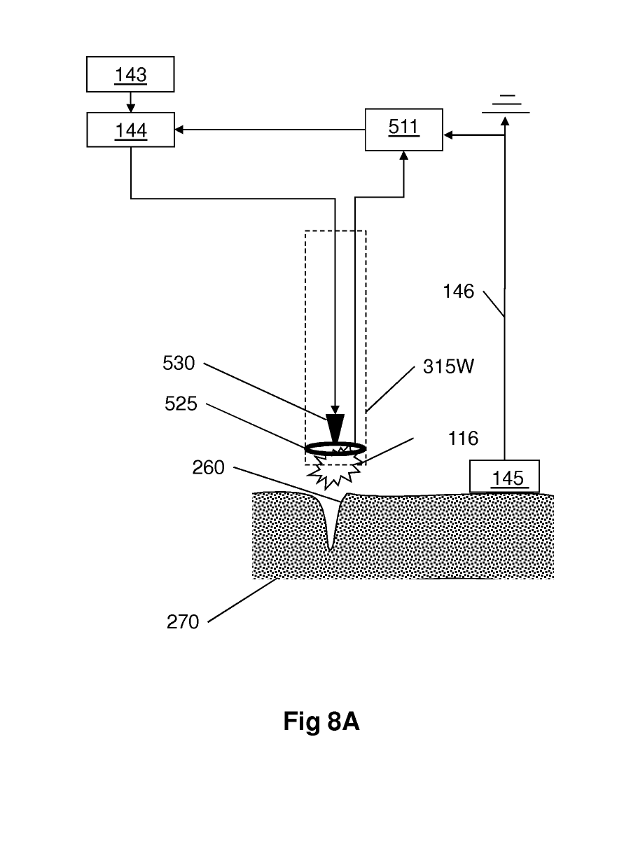

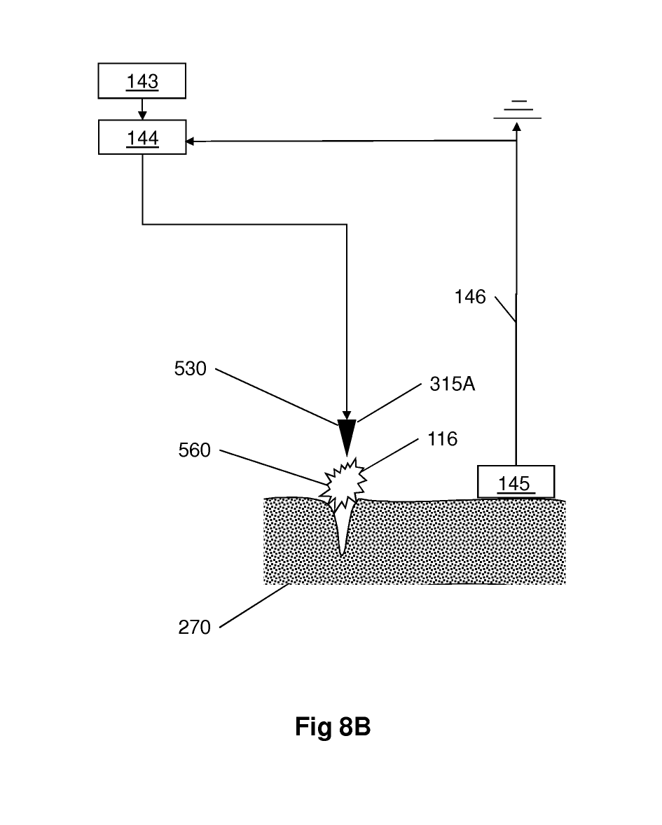

FIG. 8A schematically depicts block diagram of optional electrical circuited of a bi-polar plasma system according to an exemplary embodiment of the current invention.

This configuration may be used primarily with tissue welding plasma head such as tissue welding tip 300W.

Optional variable impedance 511 is placed in the RF electrical return line. When the impedance of variable impedance 511 is low, electrical return current is flowing primarily from central electrode 530 through ground electrode 525. Thus, the device acts as mainly bi-polar.

In contrast, when the impedance of variable impedance 511 high, the electrical return current is flowing primarily from central electrode 530 to patient's body 270 and returning via grounding electrode pad 145 electrically connected through grounding cable 146. Thus, the device acts as mainly mono-polar. When the impedance of variable impedance 511 intermediate, the device acts as a combination of bi-polar and mono-polar.

An RF forward and backwards power measurement may be done by the standard devices (dual directional coupler) which are here assumed to be part of the impedance matching circuit 144. The forward power is monitored and passes a signal to the generator power control. When the forward power exceeds a certain power (for example 10 W), the generator decreases the power and maintain a maximum power as preset.

When the patient body, electrically grounded to grounding pad is closer to the plasma tip, the plasma impedance is lower, and the power that the plasma absorbs is higher and thus the forward power shows higher readings, (or the impedance which is monitored becomes lower) this may be feedback to the controller to regulate the power to the lower power preset. An alternative possible control method, experimentally demonstrated, is “plasma current measurement” wherein a wire loop around the plasma senses the charge that passes in the plasma and points on the plasma density and plasma power.

FIG. 8B schematically depicts the electrical connections of a mono-polar plasma system according to an exemplary embodiment of the current invention.

This configuration may be used primarily with tissue ablation plasma head such as tissue ablation tip 300A.

Electrical return current is flowing from ablation electrode 530 to patient tissue 270 and returns through grounding pad 145 electrically connected to the patient's body. Thus, the device acts as mono-polar. Mono-polar plasma 116 then ablates tissue 270 creating a cut 560.

It should be noted that bi-polar tip and electrical circuit may act as mono-polar tip and electrical circuit by changing the characteristics of variable impedance 511, forcing the RF electrical circuit to close through grounding pad 145. Optionally, ablation however may be performed with a contact of electrode 530 to the tissue.

Optionally, central electrode 530 (FIG. 5a) may be slide towards the tissue (or tube 315W retracted toward the plasma head body 112), to expose the central electrode 530 when mono-polar ablation or coagulation action is needed. It should be noted that welding action and ablation or coagulation may require different RF parameters such as frequency, power and modulation.

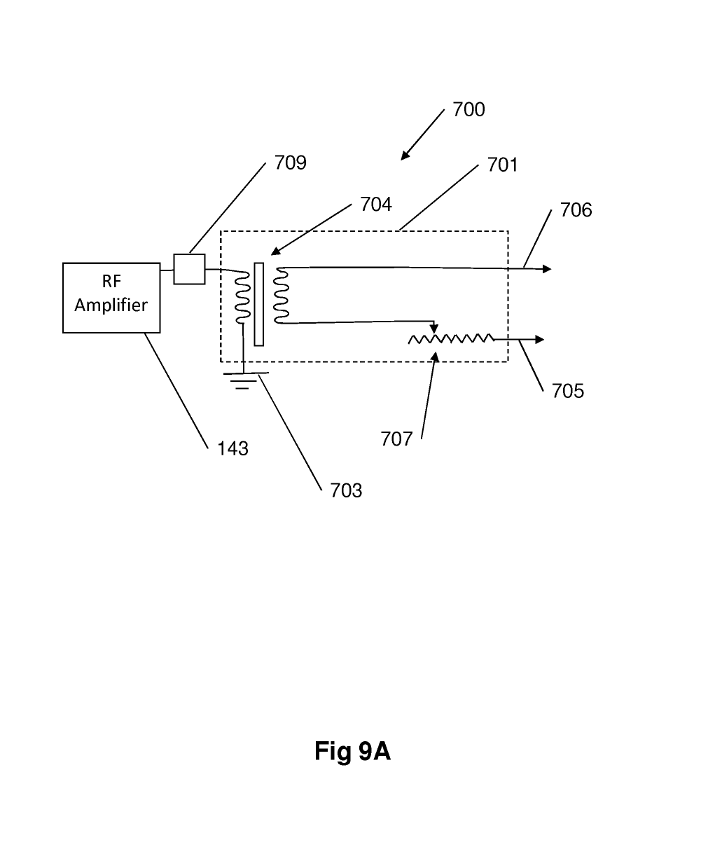

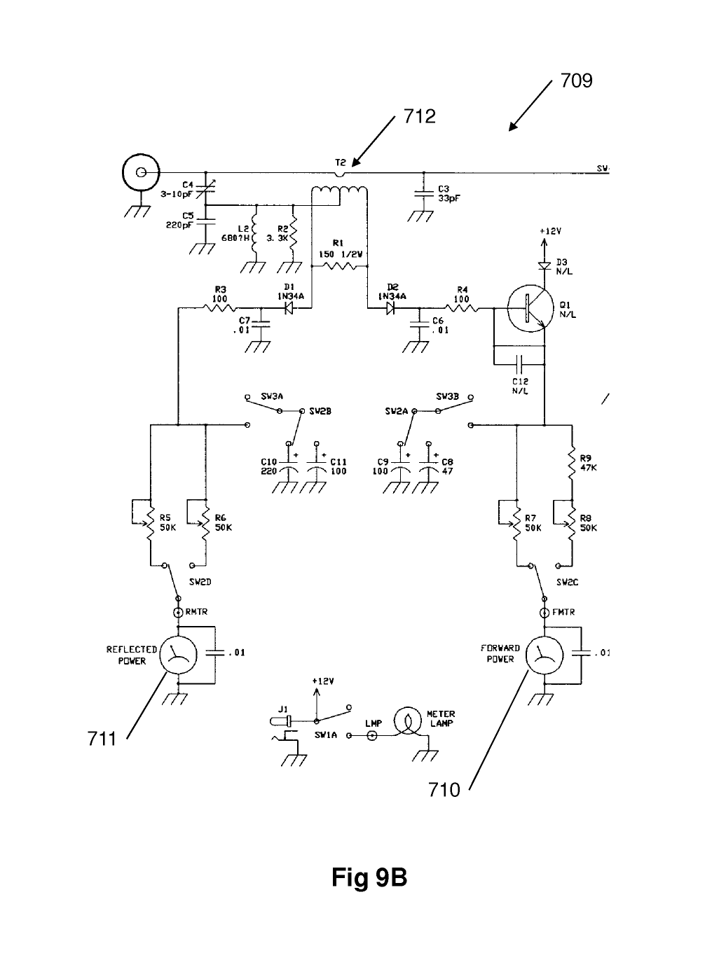

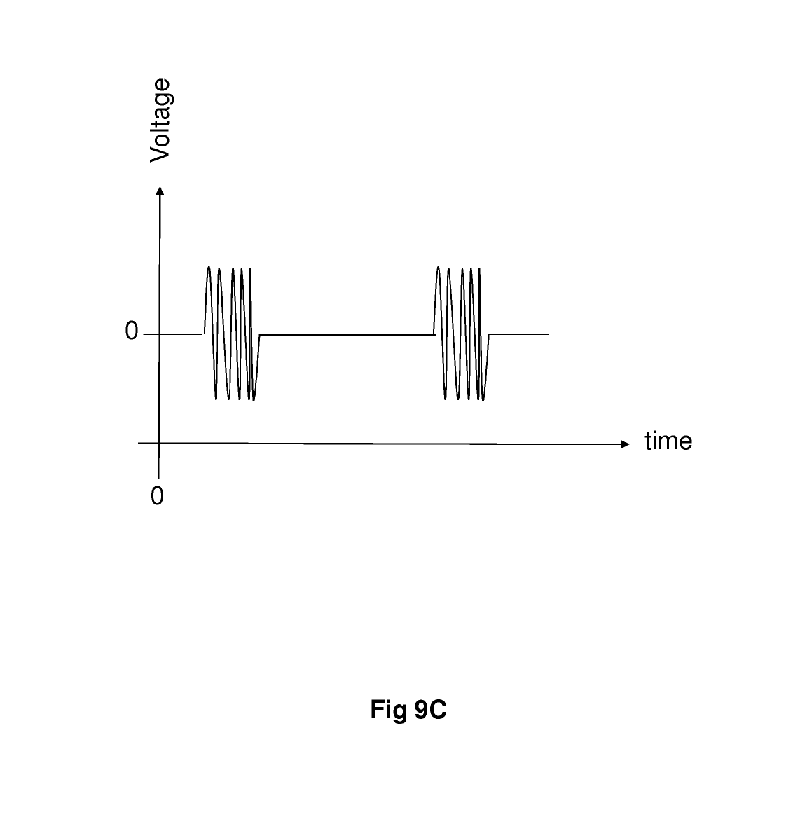

FIG. 9A schematically depicts an electric circuit 700 for driving a bipolar plasma head according to an exemplary embodiment of the invention.

Bipolar isolator 701 is inserted between RF amplifier 143 and RF cables 705 and 706 leading to a first and a second electrode (for example electrodes 417 and 414 of FIG. 3), or to the coil (for example coil 467 in FIG. 5A). It should be noted that isolator 700 may replace, or be a part of impedance matching unit 144.

As a result, the RF voltage is floating by using the transformer 704 with respect to the ground (patient's body potential) 703 and is thus between one electrode to another. This causes the plasma to be directed from the first to the second electrode and not to the ground (the patient's body) as in a uni-polar configuration.

This optional embodiment may enables determining the electric current flow of direction and instead of flowing through the patient's body to the ground electrode (as in uni-polar), it flows to the second bipolar electrode which can be inserted at a desired location.

An optional variable load such as a variable resistor 707 between the source and one of the electrodes differentiate the power transferred to the electrodes and enables transferring more power from one electrode than the other by “wasting” power on the load.

Optionally plasma parameter monitor 709 is inserted in line with the output 701 of RF amplifier 143.

Alternatively, isolator 701 and/or monitor 709 are inserted after, or integrated into impedance matching circuit 144 as seen in FIG. 1.

FIG. 9B schematically depicts electronic circuit for plasma monitor 709. In this electronic schematic, meters 710 and 711 showing transmitted and reflected power respectively receives signals from sensing coil 712.

In an exemplary embodiment of the invention, signals indicative of transmitted and/or reflected RF power are optionally digitized and transferred to processor 161 via line 148 as seen in FIG. 1 and are used for plasma monitoring and control.

By knowing the transmitted and reflected RF power it is possible to know the power deposited in the plasmas and to deduct the impedance of the treated surface or the distance to ground. If the distance to ground is known and constant, the only free parameter is the surface conductivity which is indicative of the solder denaturation state.

For example, increase of the impedance may be indicative of dehydration of the solder after it already been crossed linked by the plasma. In this case, the plasma may be turned off to prevent thermal damage to the tissue.

In some embodiments of the invention, the plasma parameter monitor such as plasma parameter monitor 709 is adapted to measure the plasma parameters and keep the plasma within a defined range such as a preset power, current, etc.

In some embodiments of the invention, the plasma parameter monitor such as plasma parameter monitor 709 is adapted to measure changes in the state of the solder applied to the tissue by monitoring changes in impedance of the plasma generation electrical path, It should be noted that both the resistance (the real part) and the induction or capacitance (the imaginary part) of impedance may be monitored. Since the tissue does not substantially change during the welding process, the impedance change reflects the change in the state of the solder and may be used as an indication of the welding process such as solidifying. The obtained signal may be used by the operator as an indication that the welding is complete and optionally may be used for automatic stopping of plasma generation for example in order to prevent overheating and thermal damage to the welding site. Similarly, the obtained signal may be used by the user to adjust the rate of plasma head motion such that even weld may be obtained with continues plasma head motion. Preferably, the distance from the plasma head to the tissue remain constant during the monitoring process.

In some embodiments, the solidifying and/or desiccation of the solder layer increases the resistivity of the already treated solder relative to the liquid solder solution, This effect may cause the plasma to be directed from a location of already welded solid solder to yet liquid solder, thus enhancing the weld uniformity.

In some embodiments of the invention, the plasma parameter monitor such as plasma parameter monitor 709 is adapted to measure the plasma parameters such as the current or the impedance and to stop the plasma generation when the current or the delivered power is below or above a threshold level, or the impedance is below or above a threshold as this conditions indicated a completion of the welding process.

For example, a short pulse of plasma may be applied for short period (for example 0.1-5 seconds) in order to sense the impedance or the current at a specific location. If the impedance is above the threshold, the plasma continues. If the impedance is still out of limits, the plasma stops for a short duration (for example 0.1-5 seconds) before another sensing pulse is generated. This process continues until the tip is located above a “non welded area” where the welding process may continue. Optionally audio or visual indication is given to the operator to indicate that the plasma head is to be moved to a new location. Optionally, plasma parameters used for sensing is at lower plasma power level than the power used for welding.

Optionally, the slope or derivative of at least one of: the impedance, power, current; or voltage change over time is monitored instead of its absolute values in order to achieve a more sensitive and robust reading, and to eliminate variation between welding operations due to free parameters change as room temperature and such. Impedance may change in the range of 10 to 1000 Ohm; power may change in the range of 0.01 to 5 W and current may change in the range of 1 to 100 mA.

Optionally, ignition of the plasma is done by applying a high voltage sinusoidal pulse of about 2 KV, and after ignition, the RF voltage is lowered to operating range of 100-500V.

In some embodiments a complex RF waveform is used, for example the RF waveform may comprise:

A sinusoidal carrier wave at frequency of 1-10 MHz which is modulated by an envelop function. The envelop function may be an on/off step function creating a train of short pulses at a specific pulse duration and duty cycle, or other envelope shape function. The envelop function may have a DC part such that it modulates the RF power without turning it off completely.

Additional signals may optionally be mixed in the final RF output such as a DC current that may be used for directing the positive and negative ions in the plasma to different direction, or other frequencies or combination thereof.

FIG. 9C schematically depicts an RF wavefunction according to an exemplary embodiment of the current invention.

The figure is not to scale, and is used as illustration of the low duty cycle train of RF pulses that enable producing stable plasma (due to the high RF voltage), yet relatively low power (due to the low duty cycle). Low duty cycle of short RF pulses, separated by longer durations of no RF excitation is preferably used in order to reduce the average RF power supplied to the plasma, thus reducing the power deposition and plasma temperature. During the pulses, the RF voltage is kept above the threshold voltage for maintaining plasma production. In some applications, quiescent time duration between pulses is selected to be shorter than the plasma lifetime, thus the plasma is not completely extinguished between pulses. Consequently, pulse voltage may be kept at, or somewhat above the threshold level require to maintain plasma instead of the higher threshold level required for plasma ignition.

The following parameters may be viewed as exemplary parameters used in a plasma hand piece that uses an RF antenna coil for plasma excitation as described in herein for example in FIG. 5B. Some parameters may be adopted for use with other plasma heads.

The antenna coil is connected to the RF circuit at one end only. This configuration allows high voltage between the electrode and the patient body and when plasma is ignited, the interaction is between the coil and the body, thus improving the tissue welding.

The RF signal is characterized by Frequency of 1-4 MHz; Modulation frequency of 200-600 Hz; and Duty cycle of 5-20%

Optionally, the antenna coil 467 is placed outside the electrically insolating tube 469 and therefore is not in a direct contact with the plasma gas 470 in the tube 466. In such a manner, the antenna coil 467 creates cold plasma instead of high temperature plasma, thus avoiding charring of the skin below the welding area.
Such a configuration enables igniting the plasma at a distance of more that 1 cm from the target tissue.
Unlike hot plasma, cold plasma does not create superficial charring and blocking of the solder top layer but rather induces deep solidifying effect of the solder. These phenomena may be explained due to lower plasma-patient voltage and so, lower ion bombardment.
The coil is used in a mono-polar configuration, for example as seen in FIGS. 1 and 8B, using electrical coupling (grounding electrode 145) between the RF circuit and the patient body.
   Electrode in Contact with Gas Vs. Dielectric Breakdown Discharge:

Most of the existing medical plasma devices use a configuration where the RF electrode is in direct contact with the gas. This configuration is called corona discharge. When using such configuration, a very superficial charring and burning is achieved. Such superficial effect is favorable for the plasma coagulation devices. Welding however, needs deep energy transfer that penetrates down to the solder's bottom and insures good “sticking” of the solder.

For welding, a combination of outer coil and un-insulated inner electrode, for example as described in U.S. Pat. No. 6,099,523, is not preferable. The combination of electrodes generates coagulation plasma with high plasma-patient voltage that induces charring of the surface. On the other hand, using the coil in a mono-polar configuration produces low power plasma which is not sufficient for tissue coagulation. In such a manner, the tissue welding does not cause collateral damage to the tissues as low energy plasma is used.

“Antenna Coil” Vs. “Resonant Coil”

A resonant coil is the common configuration that is used frequently, for example in the semiconductors industry. For example see U.S. Pat. No. 5,883,470 that describes such a resonant coil.

The resonant coil is connected to the RF source at both end (such as seen in FIG. 5A) and is in fact a part of the RF resonant circuit. The resonant coil has benefits as being almost purely inductively coupled Plasma (ICP), and usually used with high powers plasma production for example used for material processing such as in the semiconductor industries.

Resonant coil configuration is decoupled from the ground and was found to be less suitable for tissue welding because the plasma energy is coupled to the patient body. As can be understood from the disclosure herein, the welding process uses an electrical interaction between the plasma device and the patient body. Electric energy that flows from the plasma device to the solder, solidifies it. From the above, it can be understood that the plasma device needs to be electrically coupled to the solder-patient body.

FIG. 10 schematically depicts a wiper 950 for uniformly spreading solder solution on tissue according to another embodiment of the current invention.

Wiper 950 comprises a handle 951 constructed to be hand held and preferably ergonomically shaped, for example in may be bent foe ease of manipulation, have finger grips or covered with non-slip material. At the distal end, a wiper member 952 is attached. Wiper member 952 is preferably made of thin elastic material such as silicon rubber or other soft material and is used for uniformly spreading solder solution applied to a tissue or a cut in the tissue or skin.

Optionally, an indentation 954 in the edge 953 of the wiper member 952 help in leaving the desired among of solder solution, while wiping off excess solder. For example, and without limitation, the indentation in the blade id 06 mm deep and 6 mm wide.

FIG. 11A schematically depicts a roller device 970 for uniformly spreading solder solution on tissue according to another embodiment of the current invention.

Roller device 970 comprises a roller 971 that capable of rotating around an axis 972 when it is pulled in the direction 990 for example by handle 974. An solder container 976 above roller 971 contain solder solution 979. Gas pipe 977 may supply pressurized gas that forces the solder solution 979 from the solder container 976 onto the porous surface of roller 971. As roller 971 rolls over the surface of the tissue (not seen in this figure for drawing clarity), it leaves a uniform layer of solder 975.

In an exemplary embodiment, the width 978 of roller 971 is approximately 5 mm. The porous roller may have pores diameter of approximately 0.1 mm. In some embodiments, the entire roller is made of porous material such as foam. Alternatively, roller 971 is covered with a porous layer.

FIG. 11B schematically depicts a roller device 980 for uniformly spreading solder solution on tissue according to yet another embodiment of the current invention.

In contrast to roller device 970 roller device 980 comprises an solder spreader 989 connected to an solder injector 981 containing solder solution 979. Solder injector 981 is fitted with a piston 982 that pushes the solder solution from injector 981 through spreader 989 onto the surface of roller 971. Piston 982 may be activated by pressurized gas supplied by gas pipe 987. Using a sealed solder injector 981 has the advantage that roller device 980 may be operated at any orientation, for example upside down or tilted.

It should be apparent to a man skilled in the art of medical devices that other methods of rotating roller 971, for example using miniature electric motor are possible within the general scope of the current invention,

It also should be apparent to a man skilled in the art of medical devices that other methods of operating piston 982, for example using miniature electric motor or piezoelectric pusher are possible within the general scope of the current invention.

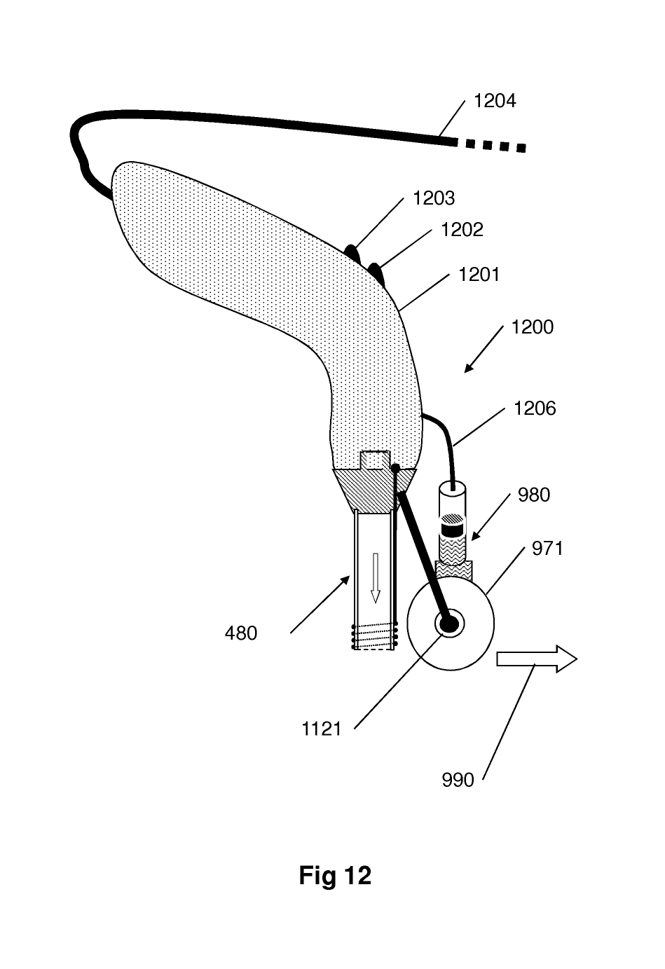

FIG. 12 schematically depicts some details of a dual-function dispenser-plasma device 1200 for both applying solder solution on tissue and performing tissue welding according to yet another exemplary embodiment of the current invention.

Dispenser-plasma device 1200 comprises a handle 1201 connected to a control box (not seen in this figure) via hose 1204. Hose 1204 supplies plasma gas and RF power for operation of a plasma head such as plasma welding tip using antenna coil 480, and optionally electric power or pressurized gas for operation of a solder dispenser such as roller device 980.

In the depicted embodiment, internal RF cable and gas pipes within the handle 1201 are not drawn for drawing clarity.

Handle 1201 may comprise an operation button or a plurality of buttons or switches such as buttons 1202 and 1203 for controlling the operation of the dispenser-plasma device 1200.

For example, button 1202 for may be used to activate solder dispensing, for example by providing pressurized gas via pipe 1206 to push solder solution onto the roller as disclosed in FIG. 11A or 1B. Alternatively, a solder dispensing method similar to injector 118 (seen in FIG. 2A) may be used. Optionally to injector 118 is operated by button 1202, for example by applying pressurized gas to push piston 230 in the injector 118, or opening valve 211.

For example button 1203 may be used for activating the plasma head.

By pressing both buttons, and moving the dispenser-plasma device 1200 in the direction 990, solder is applied to the cut in the tissue and then the applied solder is solidified by the plasma. A second layer of solder may be applied by repeating the operation. In some embodiments a motion sensor such as rotation sensor 1121 is used to measure the motion of dispenser-plasma device 1200 by detecting or measuring the rotation of roller 971. Optionally, the motion sensor is connected to a controller that activates the plasma when, or only when the dispenser-plasma device 1200 is moving over the tissue. In some embodiments, the controller also activates solder dispensing when motion is detected. Optionally, plasma power, and/or rate of solder dispensing is controlled and in relation to the speed of detected motion.

Other methods of applying a uniform layer of solder may optionally be used:

For example, solder solution may be sprayed from a pressurized container using spraying methods as known in the art. Optionally a narrow nozzle is used to aim the spray into the cut.

In some embodiments, chitosan solution or solid chitosan sheets may be used. Commercially available Chitosan comes in powder form but can be dissolved into a solution for example in acidic environment and added to the solder solution. The solution may be dried in form of sheets and used for the welding process.

The sheet may be applied on the wound, and then small amount of water or other liquid as saline solution, the patient's blood, blood plasma, or other biocompatible liquid is applied or sprayed on the sheet and partially dissolve it. Plasma beam is then directed to the partially dissolved solder or solder and Chitosan mixture thus performing the welding.

Using this method, strong weld may be achieved. Additionally, better water solubility and better process control may be achieved.

Other methods of sealing the welded area post-welding may optionally be used:

For example, adhesive tape as can be purchased in any pharmacy may be used for sealing the welding site from desiccation. A standard “steristrip” as being used in operation rooms may optionally be used for this purpose. Optionally a waterproof membrane, such as nylon may be used for better moisture retention.

Optionally moisturizing cream may be applied post-welding.

Spraying the Solder post-welding and covering with adhesive tape as can be purchased in any pharmacy may be used for sealing the welding site from desiccation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medical device for tissue welding comprising
at least one processor configured to regulate cold plasma production in a plasma head by control of an RF power source to supply RF plasma-producing power to the plasma head, the processor being configured to regulate supplied RF energy in a carrier signal with a frequency of 1 to 10 MHz, modulated at a frequency up to 600 Hz, and with a duty cycle of at least 2 to 20%.

2. (canceled)

3. The medical device of claim 1, wherein said modulated frequency occurs using on/off modulation.

4. The medical device of claim 1, wherein said plasma producing RF power further comprises a DC component.

5. The medical device of claim 1, wherein said carrier of said plasma-producing RF power is at the range of 100 to 6000 Volts.

6. The medical device of claim 1, wherein said power supply is capable of delivering to said plasma head an RF plasma ignition signal larger than 1500 Volts.

7. The medical device of claim 1, further comprising:
at least a first plasma head and a second plasma head capable of receiving gas from a gas source and RF power from an RF power source; and
a solder dispenser associated with at least one of the first and second plasma heads and configured to dispense solder during tissue welding.

8. The medical device of claim 7, wherein said solder dispenser is located between said first plasma head and said second plasma head.

9. The medical device of claim 7, further comprising at least a third plasma head.

10-46. (canceled)

47. The medical device of claim 7, wherein the solder is a bio-compatible liquid capable of solidifying in response to application of plasma, wherein said dispenser includes a container for holding said bio-compatible liquid, and the medical device further comprises a roller of porous material, for contacting tissue to be welded, receiving said bio-compatible liquid from said container, and being capable of rotating and spreading said liquid on said tissue.

48. The medical device of claim 7, further arranged to monitor an electrical impedance of the solder and to monitor at least one of power, current and voltage with respect to a solidification of said bio-compatible liquid as determined according to changes in the monitored impedance.

49. The medical device of claim 7, further comprising a motion detector configured to control operation of at least one of said plasma producing heads.

50. The medical device of claim 1, further comprising:
a plasma tip capable of receiving gas from a gas source and RF power from the RF power source, said plasma tip comprising:
a plasma tube having a proximal opening and a distal opening for receiving gas from said gas source through the proximal opening and for providing plasma through the distal opening; and
at least one electrode, for receiving RF power from said RF source, and for exciting said gas to produce said plasma,
wherein said plasma tip is configured to prevent the electrode from making direct contact with said gas thereby preventing corona plasma production while facilitating plasma production via dielectric breakdown.

51. The medical device of claim 50, further comprising a grounding electrode pad for grounding tissue.

52. The medical device of claim 50, wherein said plasma tip comprises a single electrode in a form of a coil configured to receive RF power from one end only.

53. The medical device of claim 52, wherein said plasma tube is electrically insulating, and said coil is located on the outer side of said plasma tube.

54. A tissue welding method comprising:
configuring at least one processor to regulate cold plasma production in a plasma head by controlling an RF power source to supply RF plasma-producing power to the plasma head,
configuring the processor to regulate supplied RE energy in a carrier signal with a frequency of 1 to 10 MHz, modulated at a frequency up to 600 Hz, and with a duty cycle of at least 2 to 20%, and
applying the produced regulated cold plasma to a tissue.

55. The method of claim 54, further comprising:
applying to the tissue a bio-compatible liquid capable of solidifying in response to application of plasma;
solidifying said bio-compatible liquid by applying plasma to said bio-compatible liquid; and
controlling said plasma applied to said bio-compatible liquid by:
monitoring electrical impedance in a plasma path, and monitoring at least one of power; current and voltage;
determining solidification of said bio-compatible liquid from changes in the monitored impedance; and
controlling RF power producing said plasma in response to said determined state of said bio-compatible liquid.

56. The method of claim 54, further comprising generating a first welding layer by spraying bio-compatible liquid onto the tissue; said solidifying said bio-compatible liquid using plasma; and generating a second welding layer by repeating said generating and said solidifying.

57. The method of claim 54, further comprising:
placing a sheet of dried chitosan over the tissue to be welded;
wetting said sheet of dried chitosan; and
solidifying said chitosan by said applying plasma.

58. The method of claim 54, further comprising:
applying to tissue to be welded, bio-compatible material capable of solidifying in response to application of plasma; and
solidifying said bio-compatible material by applying plasma to said bio-compatible material.
